SEQUENCE LISTING (7 sequences)

SEQ ID NO: 1 - DNA, 20 bp, synthetic construct
  source 1..20: mol_type = other DNA
gcggttctgttcccgagtgc

SEQ ID NO: 2 - DNA, 20 bp, synthetic construct
  source 1..20: mol_type = other DNA
ggcatttacaccctacgccc

SEQ ID NO: 3 - DNA, 20 bp, synthetic construct
  source 1..20: mol_type = other DNA
acacacggtgtccgaggtct

SEQ ID NO: 4 - DNA, 20 bp, synthetic construct
  source 1..20: mol_type = other DNA
gtcgtgttggagaacgtccc

SEQ ID NO: 5 - DNA, 20 bp, synthetic construct
  source 1..20: mol_type = other DNA
ggtctggccacgccgaagcc

SEQ ID NO: 6 - DNA, 20 bp, synthetic construct
  source 1..20: mol_type = other DNA
gtggggcctgcaaatatccg

SEQ ID NO: 7 - protein, 1368 aa, Streptococcus pyogenes [sequence omitted]

(19) United States
(12) Patent Application Publication
(10) Pub. No.: US 2024/0384266 A1
(43) Pub. Date: Nov. 21, 2024

(54) TREATING CANCER

(71) Applicant: Mayo Foundation for Medical Education and Research, Rochester, MN (US)

(72) Inventors: Saad J. Kenderian, Rochester, MN (US); Claudia Manriquez Roman, Rochester, MN (US); Michelle J. Cox, Rochester, MN (US); Reona Sakemura, Rochester, MN (US)

(21) Appl. No.: 18/683,697

(22) PCT Filed: Sep. 9, 2022
(86) PCT No.: PCT/US2022/076165

§ 371 Date: Feb. 14, 2024

Related U.S. Application Data
(60) Provisional application No. 63/242,265, filed on Sep. 9, 2021.

(51) Int. Cl. C12N 15/11; A61K 39/00; A61P 35/00; C12N 9/22

(52) U.S. Cl. CPC C12N 15/11; A61K 39/4611; A61K 39/4631; A61K 39/464412; A61P 35/00; C12N 9/22; C12N 2310/20

(57) ABSTRACT

This document provides methods and materials involved in treating cancer. For example, methods and materials for using chimeric antigen receptor T cells having reduced expression levels of a tumor necrosis factor receptor 2 (TNFR2) polypeptide in an adoptive cell therapy (e.g., a chimeric antigen receptor (CAR) T cell therapy) to treat a mammal (e.g., a human) having cancer are provided.

### CROSS-REFERENCE TO RELATED APPLICATIONS

This application claims the benefit of U.S. Patent Application Ser. No. 63/242,265, filed on Sep. 9, 2021. The disclosure of the prior application is considered part of (and is incorporated by reference in) the disclosure of this application.

### SEQUENCE LISTING

This application contains a Sequence Listing that has been submitted electronically as an XML file named “07039-2077WO1.XML.” The XML file, created on Sep. 7, 2022, is 9,000 bytes in size. The material in the XML file is hereby incorporated by reference in its entirety.

### TECHNICAL FIELD

This document relates to methods and materials involved in treating cancer. For example, this document provides methods and materials for using chimeric antigen receptor (CAR) T cells having reduced expression levels of a tumor necrosis factor receptor 2 (TNFR2) polypeptide in an adoptive cell therapy (e.g., a CAR T cell therapy) to treat a mammal (e.g., a human) having cancer.

### BACKGROUND INFORMATION

CAR T cell therapy has been FDA approved in certain blood cancers. However, most patients relapse in the first 1-2 years of the therapy, and CAR T cell activity is very modest in solid tumors to date.

### SUMMARY

This document provides methods and materials for generating T cells (e.g., CAR T cells) having a reduced expression level of a TNFR2 polypeptide. For example, a T cell (e.g., a CAR T cell) can be engineered to have reduced TNFR2 polypeptide expression (e.g., for use in adoptive cell therapy). In some cases, a T cell (e.g., a CAR T cell) can be engineered to knock out (KO) a nucleic acid encoding a TNFR2 polypeptide to reduce TNFR2 polypeptide expression in that T cell. This document also provides methods and materials for using T cells (e.g., CAR T cells) having a reduced expression level of a TNFR2 polypeptide. For example, T cells (e.g., CAR T cells) having a reduced level of a TNFR2 polypeptide can be administered (e.g., in an adoptive cell therapy) to a mammal having cancer to treat the mammal.

As demonstrated herein, TNFR2 KO CAR T cells produce reduced levels of a TNFR2 polypeptide. Also as demonstrated herein, TNFR2 KO CAR T cells can enhance CAR T cell function and antitumor activity. For example, enhanced CAR T cell proliferation (e.g., antigen-specific CAR T cell proliferation) and antitumor activity can be observed after depletion of TNFR2 polypeptides in CAR T cells expressing CARs targeting CD19 (CART19 cells). In some cases, TNFR2 KO CAR T cells can be incorporated into adoptive T cell therapies (e.g., CAR T cell therapies) to treat, for example, mammals having cancer.

In general, one aspect of this document features methods for making a chimeric antigen receptor T cell having a reduced level of a TNFR2 polypeptide. The methods can include, or consist essentially of, (a) obtaining a T cell having endogenous alleles encoding said TNFR2 polypeptide and expressing said TNFR2 polypeptide, (b) disrupting at least one of said endogenous alleles encoding said TNFR2 polypeptide, thereby reducing the level of expression of said TNFR2 polypeptide by said T cell, and (c) introducing nucleic acid encoding a chimeric antigen receptor into said T cell, wherein the resulting T cell is said chimeric antigen receptor T cell having a reduced level of TNFR2 polypeptide. Step (b) can be performed before step (c). Step (c) can be performed before step (b). Step (b) can include disrupting both endogenous alleles. The T cell can be obtained from a human. Step (b) can be performed ex vivo. Step (c) can be performed ex vivo. Step (b) and step (c) can both be performed ex vivo. The chimeric antigen receptor can target a tumor-associated antigen. The tumor-associated antigen can be CD19.

In another aspect, this document features methods for making a chimeric antigen receptor T cell having a reduced level of a TNFR2 polypeptide. The methods can include, or consist essentially of, (a) obtaining a T cell (i) having endogenous alleles encoding said TNFR2 polypeptide, (ii) expressing said TNFR2 polypeptide, and (iii) expressing a chimeric antigen receptor, and (b) disrupting at least one of said endogenous alleles encoding said TNFR2 polypeptide, thereby reducing the level of expression of said TNFR2 polypeptide by said T cell, wherein the resulting T cell is said chimeric antigen receptor T cell having a reduced level of TNFR2 polypeptide. The T cell can be obtained from a human. Step (b) can include disrupting both endogenous alleles. Step (b) can be performed ex vivo. The chimeric antigen receptor can target a tumor-associated antigen. The tumor-associated antigen can be CD19.

In another aspect, this document features methods for making a chimeric antigen receptor T cell having a reduced level of a TNFR2 polypeptide. The methods can include, or consist essentially of, (a) obtaining a T cell (i) having a disruption in at least one endogenous allele encoding said TNFR2 polypeptide and (ii) expressing a reduced level of said TNFR2 polypeptide as compared to a comparable T cell lacking said disruption, and (b) introducing nucleic acid encoding a chimeric antigen receptor into said T cell, wherein the resulting T cell is said chimeric antigen receptor T cell having a reduced level of TNFR2 polypeptide. The T cell can be obtained from a human. The T cell can include a disruption in both endogenous alleles. Step (b) can be performed ex vivo. The chimeric antigen receptor can target a tumor-associated antigen. The tumor-associated antigen can be CD19.

In another aspect, this document features methods for making a chimeric antigen receptor T cell having a reduced level of a TNFR2 polypeptide. The methods can include, or consist essentially of, introducing a nucleic acid construct into a T cell ex vivo, wherein said nucleic acid construct comprises: a) a nucleic acid encoding a guide RNA, wherein said guide RNA is complementary to a messenger RNA encoding said TNFR2 polypeptide; b) a nucleic acid encoding a Cas nuclease, and c) a nucleic acid encoding a chimeric antigen receptor. The guide RNA can be encoded by a nucleic acid sequence set forth in any one of SEQ ID NOs:1-6. The Cas nuclease can be a Cas9 nuclease. The nucleic acid construct can be a viral vector. The viral vector can be a lentiviral vector. The chimeric antigen receptor can target a tumor-associated antigen. The tumor-associated antigen can be CD19. The introducing step can include transduction.

In another aspect, this document features methods for making a chimeric antigen receptor T cell having a reduced level of a TNFR2 polypeptide. The methods can include, or consist essentially of, introducing a complex into a T cell ex vivo, wherein said complex comprises: a) a guide RNA, wherein said guide RNA is complementary to a messenger RNA encoding said TNFR2 polypeptide; and b) a Cas nuclease; and introducing a nucleic acid encoding a chimeric antigen receptor into said T cell ex vivo. The Cas nuclease can be Cas9 nuclease. The complex can be a ribonucleoprotein. The chimeric antigen receptor can target a tumor-associated antigen. The tumor-associated antigen can be CD19. The introducing steps can include electroporation.

In another aspect, this document features T cell comprising (a) a disruption in at least one endogenous allele encoding a TNFR2 polypeptide and (b) nucleic acid encoding a chimeric antigen receptor, wherein said T cell expresses a reduced level of said TNFR2 polypeptide as compared to a comparable T cell lacking said disruption, and wherein said T cell expresses said chimeric antigen receptor. The T cell can be obtained from a human. The T cell can include a disruption in both endogenous alleles. The chimeric antigen receptor can target a tumor-associated antigen. The tumor-associated antigen can be CD19. The T cell have improved antitumor activity as compared to said comparable T cell lacking said disruption.

In another aspect, this document features methods for treating a mammal having cancer. The methods can include, or consist essentially of, administering, to a mammal having cancer, a composition comprising a T cell having a reduced level of a TNFR2 polypeptide. The composition can include from about 0.5×10⁶ to 10×10⁶ of said T cells per kg body weight of said mammal. The mammal can be a human. The cancer can be a lymphoma (e.g., a diffuse large B cell lymphoma). The cancer can be a leukemia (e.g., an acute lymphoblastic leukemia). The chimeric antigen receptor can target a tumor-associated antigen. The tumor-associated antigen can be CD19.

In another aspect, this document features methods for treating a mammal having cancer. The methods can include, or consist essentially of, administering chimeric antigen receptor T cells having a reduced level of a TNFR2 polypeptide to a mammal having cancer. The mammal can be a human. The cancer can be a lymphoma (e.g., a diffuse large B cell lymphoma). The cancer can be a leukemia (e.g., an acute lymphoblastic leukemia). The chimeric antigen receptor can target a tumor-associated antigen. The tumor-associated antigen can be CD19.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. FIG. 1A. There no significant differences in the expression of death receptors on resting CART cells between responders (R) and non-responders (NR) from Zuma-1 clinical trial. Expression of death receptors (Fas, FasL, TRAIL and TRAIL-R2) was measured on CART19 cells from R and NR at baseline (ns=not significant: t-test). FIGS. 1B-1C). Activated CART cells with CD19+ cell lines from non-responders have higher levels of expression of death receptors in comparison to non-responders from the Zuma-1 clinical trial. CART19 cells from NR and R were co-cultured with irradiated Nalm6 (CD19⁺) cells and expression of death receptors TRAIL (FIG. 1B) and Fas: FasL ration (FIG. 1C) was analyzed via flow cytometry after 24 hrs (*p<0.05, **p<0.01, ****p<0.0001; two-way ANOVA).

FIGS. 2A-2C. There is a strong association between an activated phenotype (characterized by high levels of Fas-L, TRAIL-L and TNFR2 expression) with more terminally differentiated CART cells (low levels of CCR7) in non-responders from the Zuma-1 clinical trial. Pearson correlation between responders and non-responders comparing death receptors at baseline (FasL (FIG. 2A) and TRAIL-L (FIG. 2B)) or after 24 hour activated (TNFR2 (FIG. 2C)).

FIG. 3. CART19 cells undergo more apoptosis upon antigen-specific stimulation in comparison to T-cell receptor (TCR) stimulation over time. CART19 cells were co-cultured with either PMA/Ionomycin, CD3/CD28 beads (TCR stimulation) or CD19⁺ cell line Nalm6. Flow cytometric staining for Annexin V and 7-AAD is performed at 0 and 2 hours (**p<0.01, ****p<0.0001; two-way ANOVA).

FIG. 4. There is a strong association between TNFR2 expression, effector CART phenotype, and lack of response based on clinical samples from Zuma-1 clinical trial. Schematic representation showing that TNFR2 expression is inversely related to CCR7, showing consistency between R and NR from the clinical trial data.

FIG. 5. Activated CART19 upregulated Fas, FasL, TRAIL, and TRAIL-R. CART19 cells from healthy donor were co-cultured with irradiated Nalm6 (CD19⁺) and expression of death receptors was measured by flow cytometry at baseline and after 48 hours (*p<0.05, **p<0.01, ***p<0.001, ****p<0.0001; two-way ANOVA).

FIG. 6. TNFR2 is persistently elevated, but not other death receptors, on CART19 cells using an extended in vitro culture model. CART19 cells from healthy donors were co-cultured with irradiated CD19⁺ Jeko-1 cells and repeatedly stimulated at days 0, 2, 4, 6 and 7. Flow cytometric analysis was done in order to measure levels of death receptors during each time point (ns=not significant, **p<0.01 ****p<0.0001; two-way ANOVA).

FIG. 7. TNFR1, but not TNFR2 is highly upregulated upon CART19 antigen-specific stimulation. CART19 cells were co-cultured with irradiated Nalm6 (CD19⁺) cells. Flow cytometric analysis was performed in order to measure TNFR1 and TNFR2 levels at days 0,1,3 & 5 (ns=not significant, ****p<0.0001; two-way ANOVA).

FIGS. 8A-8B. Generation of TNFR2^{ko}CART19 cells using CRISPR/Cas9. FIG. 8A shows exemplary nucleic acid sequences (SEQ ID NOs:1-3) that can encode a gRNA that can target TNFR2. FIG. 8B shows an exemplary lentiviral vector (pLentiCRISPRv2 model) that includes a SpCas9+gRNA expression cassette and a puromycin resistance gene.

FIG. 9. A schematic showing an exemplary method of TNFR2^{k/o} CART19 production from human healthy donors.

FIGS. 10A-10B. FIG. 10A. Representative figure showing levels of TNFR2 from CART19 (ctrigRNA) and CART19 TNFR2^{k/o} cells at day 6 of production. FIG. 10B. Representative TIDE (Tracking of Indels by Decomposition) sequence analysis used to verify genomic alteration on CART19 TNFR2^{k/o} cells.

FIGS. 11A-11C. TNFR2^{k/o} CART19 cells showed decreased levels of T cell activation markers in comparison to CART19 (ctrlgRNA) cells. TNFR2^{k/o} or ctrlgRNA CART19 cells were co-cultured with irradiated Nalm6 (CD19⁺) cell lines. Flow cytometric staining was performed at baseline (0 hours) and after 24 hours in order to measure CD25 (FIG. 11A), CD69 (FIG. 11B) and CD45 (FIG. 11C) (*p<0.05; t-test).

FIG. 12. TNFR2^{k/o} CART19 cells are less apoptotic than TNFR2wt CART19 cells upon stimulation of their CAR via CD19⁺ cells line Nalm6. TNFR2^{k/o} CART19 and TNFR2^{wt} CART19 cells are co-cultured with CD19⁺ cell line Nalm6. Flow cytometry analysis was performed to measure apoptotic cells (Annexin⁺, 7AAD⁻) at 2 hours and 4 hours. **p<0.01, ***p<0.001, ****p<0.0001; two-way ANOVA.

FIGS. 13A-13B. TNFR2k/o CART19 cells showed enhanced antigen-specific proliferation (FIG. 13A) and cytotoxicity (FIG. 13B) in comparison to CART19 (ctrlgRNA) cells. CART19 (ctrlgRNA) or TNFR2k/o CART19 cells were co-cultured with irradiated Nalm6 (CD19+) cells. PMA/lonomycin or media alone. Flow cytometry staining was performed in to measure absolute number of CD3+ cells (FIG. 13A). CART19 (ctrlgRNA) or TNFR2k/o CART19 cells were co-cultured with luciferase+ Nalm6 (CD19+) cells and cytotoxicity after 48 hours (FIG. 13B). *p<0.05, **p<0.01; two-way ANOVA.

FIG. 14. A schematic showing an exemplary in vivo xenograft model for comparing CART19 vs. CART19 TNFR2^{ko} cells.

FIGS. 15A-15C. TNFR2^{k/o} CART19 cells showed improved CART cell expansion, enhanced anti-tumor activity and survival in vivo in comparison to CART19 (ctrlgRNA) cells. NSG mice were engrafted with JeKo-1 and then randomized to receive either CART19 (ctrlgRNA) or TNFR2^{k/o} CART19 cells. Bioluminescence was measured once a week to assess burden of disease. Tail bleeding was done once a week in order to assess CART cell expansion in vivo.

### DETAILED DESCRIPTION

This document provides methods and materials for generating T cells (e.g., CAR T cells) having a reduced expression level of a TNFR2 polypeptide. In some cases, a T cell (e.g., a CAR T cell) can be engineered to KO a nucleic acid encoding a TNFR2 polypeptide to reduce TNFR2 polypeptide expression in that T cell (e.g., as compared to a comparable T cell that is not engineered to KO a nucleic acid encoding a TNFR2 polypeptide). A T cell that is engineered to KO a nucleic acid encoding a TNFR2 polypeptide can also be referred to herein as a TNFR2 KO T cell, a TNFR2^{k/o} T cell, or a TNFR2^{KO} T cell.

The term “reduced level” as used herein with respect to an expression level of a TNFR2 polypeptide refers to any level that is lower than a reference expression level of the TNFR2 polypeptide. The term “reference level” as used herein with respect to a TNFR2 polypeptide refers to the level of that TNFR2 polypeptide typically observed in a sample (e.g., a control sample) from one or more mammals (e.g., humans) not engineered to have a reduced expression level of that TNFR2 polypeptide as described herein. Control samples can include, without limitation, T cells that are wild-type T cells (e.g., T cells that are not TNFR2 KO T cells). In some cases, a reduced expression level of a TNFR2 polypeptide can be an undetectable level of that TNFR2 polypeptide. In some cases, a reduced expression level of a TNFR2 polypeptide can be an eliminated level of that TNFR2 polypeptide.

In some cases, a T cell having (e.g., engineered to have) a reduced level of a TNFR2 polypeptide can have enhanced CAR T cell function such as improved antitumor activity, improved proliferation, reduced apoptosis, improved cell killing, improved cytokine production, less exhaustion susceptibility, improved antigen specific effector functions, improved persistence, and improved differentiation (e.g., as compared to a CAR T cell that is not engineered to have a reduced level of a TNFR2 polypeptide as described herein).

A T cell having (e.g., engineered to have) a reduced expression level of a TNFR2 polypeptide such as a TNFR2 KO T cell can be any appropriate T cell. A T cell can be a naïve T cell. Examples of T cells that can be engineered to have a reduced expression level of a TNFR2 polypeptide as described herein include, without limitation, cytotoxic T cells (e.g., CD4⁺ CTLs and/or CD8⁺ CTLs), stem cell memory t cells, natural killer T (NKT) cells, and invariant NKT (INKT) cells. For example, a T cell that can be engineered to have a reduced level of a TNFR2 polypeptide as described herein can be a CAR T cell. In some cases, one or more T cells designed to have a reduced level of a TNFR2 polypeptide can be T cells that were obtained from a mammal (e.g., a mammal having cancer) that is to be treated with those T cells designed to have a reduced level of a TNFR2 polypeptide. For example, T cells can be obtained from a mammal to be treated with the materials and method described herein.

A T cell having (e.g., engineered to have) a reduced expression level of a TNFR2 polypeptide such as a TNFR2 KO T cell can be generated using any appropriate method. In some cases, a T cell (e.g., a CAR T cell) can be engineered to KO a nucleic acid encoding a TNFR2 polypeptide to reduce TNFR2 polypeptide expression in that T cell. In some cases, at least one endogenous allele of a nucleic acid encoding a TNFR2 polypeptide can be disrupted (e.g., knocked out) to generate a T cell (e.g., a CAR T cell) having a reduced expression level of a TNFR2 polypeptide. In some cases, both endogenous alleles of a nucleic acid encoding a TNFR2 polypeptide can be disrupted (e.g., knocked out) to generate a T cell (e.g., a CAR T cell) having a reduced expression level of a TNFR2 polypeptide.

In some cases, when a T cell (e.g., a CAR T cell) is engineered to KO a nucleic acid encoding a TNFR2 polypeptide to reduce expression of that TNFR2 polypeptide in that T cell, any appropriate method can be used to KO a nucleic acid encoding that TNFR2. Examples of techniques that can be used to knock out a nucleic acid sequence encoding a TNFR2 polypeptide include, without limitation, gene editing, homologous recombination, non-homologous end joining, microhomology end joining, and base pair editing. For example, gene editing (e.g., with engineered nucleases) can be used to KO a nucleic acid encoding a TNFR2 polypeptide. Nucleases useful for genome editing include, without limitation, CRISPR-associated (Cas) nucleases, zinc finger nucleases (ZFNs), transcription activator-like effector (TALE) nucleases, and homing endonucleases (HE; also referred to as meganucleases).

In some cases, a clustered regularly interspaced short palindromic repeat (CRISPR)/Cas system can be used (e.g., can be introduced into one or more T cells) to KO a nucleic acid encoding a TNFR2 polypeptide. CRISPR/Cas molecules are components of a prokaryotic adaptive immune system that is functionally analogous to eukaryotic RNA interference, using RNA base pairing to direct nucleic acid cleavage resulting in double stranded breaks (DSBs) about 3-4 nucleotides upstream of a protospacer adjacent motif (PAM) sequence (e.g., NGG). Directing nucleic acid DSBs with the CRISPR/Cas system requires two components: a Cas nuclease, and a guide RNA (gRNA) targeting sequence directing the Cas to cleave a target DNA sequence (Makarova et al., Nat Rev Microbiol, 9(6):467-477 (2011); and Jinek et al., Science, 337(6096):816-821 (2012)).

A CRISPR/Cas system used to KO a nucleic acid encoding a TNFR2 polypeptide can include any appropriate gRNA. In some cases, a gRNA can be complementary to a nucleic acid encoding a TNFR2 polypeptide (e.g., a TNFR2 mRNA). Examples of nucleic acid sequences that can encode a gRNA that is specific to a nucleic acid encoding a TNFR2 polypeptide include, without limitation, GCGGTTCTGTTCCCGAGTGC (SEQ ID NO:1), GGCATTTACACCCTACGCCC (SEQ ID NO:2), ACACACGGTGTCCGAGGTCT (SEQ ID NO: 3), GTCGTGTTGGAGAACGTCCC (SEQ ID NO:4), GGTCTGGCCACGCCGAAGCC (SEQ ID NO:5), and GTGGGGCCTGCAAATATCCG (SEQ ID NO:6).

A CRISPR/Cas system used to KO a nucleic acid encoding a TNFR2 polypeptide can include any appropriate Cas nuclease. Examples of Cas nucleases include, without limitation, Cas1, Cas2, Cas3, Cas9, Cas10, and Cpf1. In some cases, a Cas component of a CRISPR/Cas system designed to KO a nucleic acid encoding a TNFR2 polypeptide can be a Cas9 nuclease. For example, the Cas9 nuclease of a CRISPR/Cas9 system described herein can be a Streptococcus pyogenes Cas9 (spCas9). In some cases, a spCas9 can have an amino acid sequence set forth in SEQ ID NO:7.

SEQ ID NO: 7
MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD

In some cases, a Cas nuclease can be as described elsewhere (see, e.g., Sterner et al., Blood, 133(7):697-709 (2019); Sterner et al., J. Vis. Exp., 2019(149); and Liu et al., Cell Res., 27(1):154-7) (2017)).

Components of a CRISPR/Cas system (e.g., a gRNA and a Cas nuclease) used to KO a nucleic acid encoding a TNFR2 polypeptide can be introduced into one or more T cells (e.g., CAR T cells) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA (e.g., a gRNA sequence specific to a nucleic acid encoding a TNFR2 polypeptide) and a nucleic acid encoding at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA (e.g., a gRNA sequence specific to a nucleic acid encoding a TNFR2 polypeptide) and at least one Cas nuclease (e.g., a Cas9 nuclease) can be introduced into one or more T cells.

In some cases, when components of a CRISPR/Cas system (e.g., a gRNA and a Cas nuclease) are introduced into one or more T cells as nucleic acid encoding the components (e.g., nucleic acid encoding a gRNA and nucleic acid encoding a Cas nuclease), the nucleic acid can be any appropriate form. For example, a nucleic acid can be a construct (e.g., an expression construct). A nucleic acid encoding at least one gRNA and a nucleic acid encoding at least one Cas nuclease can be on separate nucleic acid constructs or on the same nucleic acid construct. In some cases, a nucleic acid encoding at least one gRNA and a nucleic acid encoding at least one Cas nuclease can be on a single nucleic acid construct. A nucleic acid construct can be any appropriate type of nucleic acid construct. Examples of nucleic acid constructs that can be used to express at least one gRNA and/or at least one Cas nuclease include, without limitation, expression plasmids and viral vectors (e.g., lentiviral vectors). In cases where a nucleic acid encoding at least one gRNA and a nucleic acid encoding at least one Cas nuclease are on separate nucleic acid constructs, the nucleic acid constructs can be the same type of construct or different types of constructs. In some cases, a nucleic acid encoding at least one gRNA sequence specific to a nucleic acid encoding a TNFR2 polypeptide and a nucleic acid encoding at least one Cas nuclease can be on a single lentiviral vector. For example, a lentiviral vector encoding at least one gRNA sequence specific to a nucleic acid encoding TNFR2 polypeptide (e.g., a lentiviral vector including a nucleic acid sequence that can encode a gRNA that is specific to a nucleic acid encoding a TNFR2 polypeptide such as a nucleic acid sequence set forth in any one of SEQ ID NOs:1-6) and encoding at least one Cas9 nuclease can be used in ex vivo engineering of T cells to have a reduced expression level of that TNFR2 polypeptide.

In some cases, components of a CRISPR/Cas system (e.g., a gRNA and a Cas nuclease) can be introduced directly into one or more T cells (e.g., as a gRNA and/or as Cas nuclease). A gRNA and a Cas nuclease can be introduced into the one or more T cells separately or together. In cases where a gRNA and a Cas nuclease are introduced into the one or more T cells together, the gRNA and the Cas nuclease can be in a complex. When a gRNA and a Cas nuclease are in a complex, the gRNA and the Cas nuclease can be covalently or non-covalently attached. In some cases, a complex including a gRNA and a Cas nuclease also can include one or more additional components. Examples of complexes that can include components of a CRISPR/Cas system (e.g., a gRNA and a Cas nuclease) include, without limitation, ribonucleoproteins (RNPs) and effector complexes (e.g., containing a CRISPR RNAs (crRNAs) a Cas nuclease). For example, at least one gRNA and at least one Cas nuclease can be included in a RNP. In some cases, a RNP including at least one gRNA sequence specific to a nucleic acid encoding a TNFR2 polypeptide (e.g., a RNP including at least one nucleic acid sequence that can encode a gRNA that is specific to a nucleic acid encoding a TNFR2 polypeptide such as a nucleic acid sequence set forth in any one of SEQ ID NOs:1-6) and at least one Cas9 nuclease can be used in ex vivo engineering of T cells to have a reduced level of a TNFR2 polypeptide.

Components of a CRISPR/Cas system (e.g., a gRNA and a Cas nuclease) used to KO a nucleic acid encoding a TNFR2 polypeptide can be introduced into one or more T cells (e.g., CAR T cells) using any appropriate method. A method of introducing components of a CRISPR/Cas system into a T cell can be a physical method. A method of introducing components of a CRISPR/Cas system into a T cell can be a chemical method. A method of introducing components of a CRISPR/Cas system into a T cell can be a particle-based method. Examples of methods that can be used to introduce components of a CRISPR/Cas system into one or more T cells include, without limitation, electroporation, transfection (e.g., lipofection), transduction (e.g., viral vector mediated transduction), microinjection, nucleofection, cell penetrating polymers, cell squeezing, and nanoparticles (e.g., gold nanoparticles, lipid nanoparticles, and polymeric nanoparticles). In some cases, when components of a CRISPR/Cas system are introduced into one or more T cells as nucleic acid encoding the components, the nucleic acid encoding the components can be transduced into the one or more T cells. For example, a lentiviral vector encoding at least one gRNA sequence specific to a nucleic acid encoding a TNFR2 polypeptide (e.g., a lentiviral vector including a nucleic acid sequence that can encode a gRNA that is specific to a nucleic acid encoding a TNFR2 polypeptide such as a nucleic acid sequence set forth in any one of SEQ ID NOs:1-6) and at least one Cas9 nuclease can be transduced into T cells (e.g., ex vivo T cells). In some cases, when components of a CRISPR/Cas system are introduced directly into one or more T cells, the components can be electroporated into the one or more T cells. For example, a RNP including at least one gRNA sequence specific to a nucleic acid encoding a TNFR2 polypeptide (e.g., a RNP including at least one nucleic acid sequence that can encode a gRNA that is specific to a nucleic acid encoding a TNFR2 polypeptide such as a nucleic acid sequence set forth in any one of SEQ ID NOs:1-6) and at least one Cas9 nuclease can be electroporated into T cells (e.g., ex vivo T cells). In some cases, components of a CRISPR/Cas system can be introduced ex vivo into one or more T cells. For example, ex vivo engineering of T cells have a reduced level of TNFR2 polypeptide can include transducing isolated T cells with a lentiviral vector encoding components of a CRISPR/Cas system. For example, ex vivo engineering of T cells having reduced levels of a TNFR2 polypeptide can include electroporating isolated T cells with a complex including components of a CRISPR/Cas system. In cases where T cells are engineered ex vivo to have a reduced level of a TNFR2 polypeptide, the T cells can be obtained from any appropriate source (e.g., a mammal such as the mammal to be treated or a donor mammal, or a cell line).

In some cases, a ZFN system can be used (e.g., can be introduced into one or more T cells) to KO a nucleic acid encoding a TNFR2 polypeptide. ZFNs are artificial restriction enzymes generated by fusing a zinc finger DNA-binding domain to a DNA-cleavage domain. The DNA-binding domains of individual ZFNs typically contain between three and six individual zinc finger repeats and can each recognize between 9 and 18 basepairs. Zinc finger domains can be engineered to target specific desired DNA sequences (see, e.g., Durai et al., Nucleic Acids Res., 33(18):5978-5990 (2005); Bibikova et al., Science, 300(5620):764 (2003); Mandell et al., Nucleic Acids Res., 34:W516-23 (2006); and Porteus et al., Nat. Biotechnol., 23(8):967-973 (2005)). For example, an engineered zinc finger DNA-binding domain can be fused to a DNA-cleavage domain to create a ZFN that can create nucleic acid

DSBs at or near the sequence targeted by the zinc finger DNA-binding domain. A ZFN system can include any appropriate nuclease. In some cases, a nuclease can be a non-specific nuclease. In some cases, a nuclease can function as a dimer. For example, when a nuclease that functions as a dimer is used, a highly site-specific restriction enzyme can be created. For example, a FokI nuclease can used in a ZFN system described herein (see, e.g., Kim et al., Proc. Natl. Acad. Sci. USA, 93(3):1156-1160 (1996); Guo et al., J. Mol. Bio., 400 (1): 96-107 (2010); and Bitinaite et al., Proc. Natl. Acad. Sci. USA, 95(18):10570-10575 (1998)).

In some cases, a TALEN system can be used (e.g., can be introduced into one or more T cells) to KO a nucleic acid encoding a TNFR2 polypeptide. Transcription activator-like (TAL) effectors are found in plant pathogenic bacteria of the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al., Science 318:648-651, 2007; Sugio et al., Proc Natl Acad Sci USA 104:10720-10725, 2007; and Römer et al., Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site selection and engineering of new TALENs with binding specificity for the selected sites. For example, an engineered TAL effector DNA binding domain targeting sequence can be fused to a nuclease to create a TALEN that can create nucleic acid DSBs at or near the sequence targeted by the TAL effector DNA binding domain. Directing nucleic acid DSBs with the TALEN system requires two components: a nuclease, and TAL effector DNA-binding domain directing the nuclease to a target DNA sequence (see, e.g., Schornack et al., J. Plant Physiol. 163:256, 2006). A TALEN system can include any appropriate nuclease. In some cases, a nuclease can be a non-specific nuclease. In some cases, a nuclease can function as a dimer. For example, when a nuclease that functions as a dimer is used, a highly site-specific restriction enzyme can be created. For example, each nuclease monomer can be fused to a TAL effector sequence that recognizes a different DNA target sequence, and only when the two recognition sites are in close proximity do the inactive monomers come together to create a functional enzyme. Examples of nucleases that can used in a TALEN system described herein include, without limitation, FokI, HhaI, HindIII, NotI, BbvCI, EcoRI, BglI, and AlwI. For example, a nuclease of a TALEN system can include a FokI nuclease (see, e.g., Kim et al. (1996) Proc. Natl. Acad. Sci. USA 93:1156-1160).

In some cases, a T cell (e.g., a CAR T cell) can be treated with one or more inhibitors of TNFR2 polypeptide expression to reduce TNFR2 polypeptide expression in that T cell (e.g., as compared to a T cell that was not treated with one or more inhibitors of TNFR2 polypeptide expression). An inhibitor of TNFR2 polypeptide expression can be any appropriate inhibitor. Example of inhibitors of TNFR2 polypeptide expression include, without limitation, nucleic acid molecules designed to induce RNA interference (e.g., a siRNA molecule or a shRNA molecule), antisense molecules, and miRNAs.

A T cell having (e.g., engineered to have) a reduced expression level of a TNFR2 polypeptide can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed in a T cell having reduced expression of a TNFR2 polypeptide as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alphafetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, C-met, mesothelin, CS-1, and FAP. For example, a T cell having a reduced level of a TNFR2 polypeptide can be designed to express an antigen receptor targeting CD19.

In some cases, a CAR can be designed to include a single chain antibody (e.g., a scFv) targeting a tumor antigen. For example, a CAR can be designed to include a single chain antibody as set forth in Table 1.

TABLE 1
Exemplary CARs for targeting tumor antigens.

| Antigen | scFv antibody name | Reference(s) |
|---|---|---|
| CD19 | FMC63 | Milone et al., Mol. Ther., 17(8): 1453-64 (2009) (PMID: 26330164) |
| | MOR208 | Kellner et al., Leukemia, 27(7): 1595-8 (2013) (PMID: 23277329) |
| | Humanized scFv | International Patent Application Publication No. WO2015157252 |
| | 4G7 | EP Patent No. EP2997141 |
| | Low affinity scFv | Chinese Patent No. CN107406517 |
| MUC-1 | 5E5 | Posey et al., Immunity, 45(5): 947-948 (2016) (PMID: 27851918) |
| HER-2 | 4D5 | Ohnishi et al., Br. J. Cancer, 71(5): 969-73 (1995) (PMID: 7734322) |
| | | Forsberg et al., Cancer Res., 79(5): 899-904 (2019) (PMID: 30622115) |
| | | Nellan et al., J. Immunother. Cancer, 6(1): 30 (2018) (PMID: 29712574) |
| | | Priceman et al., Clin Cancer Res., 24(1): 95-105 (2018) (PMID: |
| | | 29061641) |
| | FRP5 | Bielamowics et al., Neuro. Oncol., 20(4): 506-518 (2018) (PMID: |
| | | 29016929) |
| EGFR | M27 | Jiang et al., Cancer Immunol. Res., 6(11): 1314-1326 (2018) (PMID: |
| | | 30201736) |
| | Cetuximab | Caruso et al., Cancer Res., 75(17): 3505-18 (2015) (PMID: 26330164) |
| Folate receptor alpha | C4 based | Ao et al., J. Immunother., 42(8): 284-296 (2019) (PMID: 31261167) |
| | MOv19 | Song et al., J. Hematol. Oncol., 9(1): 56 (2016) (PMID: 27439908) |
| Mesothelin | SS1 | Haas et al., Mol. Ther., S1525-0016(19)30328-4 (2019) (PMID: |
| | | 31420241) |
| | M clone | Adusumilli et al., Sci. Transl. Med., 6(261): 261ra151 (2014) (PMID: |
| | | 25378643) |
| AFP | ET1402L1 | Liu et al., Clin. Cancer Res., 23(2): 478-488 (2017) (PMID: 27535982) |
| CEA | Anti-CEA scFv | Chi et al., Cancer Med., 8(10): 4753-4765 (2019) (PMID: 31237116) |
| | CEACAM5 | Thistlethwaite et al., Cancer Immunol. Immunother., 66(11): 1425-1436 |
| | | (2017) (PMID: 28660319) |
| | hMN14 | Katz et al., Clin. Cancer Res., 21(14): 3149-59 (2015) (PMID: |
| | | 25850950) |
| CD123 | 22172, 22176 | Gill et al., Blood, 123(15): 2343-54 (2014) (PMID: 24596416) |
| | Humanized scFv | US Patent Application Publication No. 2016/0068601 |
| | Humanized scFv | EP Patent No. EP2968415 |
| | Tagrazofusp based | Pemmaraju et al. N. Engl. J. Med., 380(17): 1628-1637 (2019) (PMID: |
| | | 31018069) |
| CD33 | MY96 | Kenderian et al., Leukemia, 29(8): 1637-47 (2015) (PMID: 25721896) |
| | Humanized scFv | International Patent Application Publication No. WO2016014576 |
| CLEC12A | Humanized scFv | US Patent Application Publication No. US20160051651 |
| | CLL1 scFv | International Patent Application Publication No. WO2016120219 |
| CD22 | M0971 | Fry et al., Nat. Med., 24(1): 20-28 (2018) (PMID: 29155426) |
| | Humanized scFv | International Patent Application Publication No. WO/2016/164731 |
| | clones | |
| | Inotuzumab based | Kantarjian et al., N. Engl. J. Med., 375(8): 740-53 (2016) (PMID: |
| | | 27292104) |
| | Moxetumomab based | Kreitman et al., Leukemia, 32(8): 1768-1777 (2018) (PMID: 30030507) |
| CD20 | Rituximab | Zhang et al., Signal Transduct. Target Ther., 1: 16002 (2016) (PMID: |
| | | 29263894) |
| | Leu 16 | Lee et al., J. Immunother., 41(1): 19-31 (2018) (PMID: 29176334) |
| | CD20 scFvs | International Patent Application Publication No. WO/2016/164731 |
| BCMA | BCMA-02 | Raje et al., N. Engl. J. Med., 380(18): 1726-1737 (2019) (PMID: |
| | | 31042825) |
| | LCAR38 | Zhao et al., J. Hematol. Oncol., 11(1): 141 (2018) (PMID: 30572922) |
| | BCMA scFv | Smith et al., Cancer Immunol. Res., 7(7): 1047-1053 (2019) (PMID: |
| | | 31113804) |
| | Biepitopic | Xu et al., Proc. Natl. Acad. Sci. USA, 116(19): 9543-9551 (2019) |
| | | (PMID: 30988175) |
| | NVS BCMA | Cohen et al., J. Clin. Invest., 129(6): 2210-2221 (2019) (PMID: |
| | | 30896447) |
| CS-1 | CS1R | Wang et al., Clin. Cancer Res., 24(1): 106-119 (2018) (PMID: |
| | | 29061640) |
| | CS1 ScFv | Chu et al., Leukemia, 28(4): 917-27 (2014) (PMID: 24067492) |
| | Elotuzumab based | Dimopoulos et al., N. Engl. J. Med., 379(19): 1811-1822 (2018) (PMID: |
| | | 30403938) |
| CD138 | ScFv | Sun et al., Oncotarget., 10(24): 2369-2383 (2019) (PMID: 31040928) |
| CD44v6 | Humanized scFv | Leuci et al., Oncoimmunology, 7(5): e1423167 (2018) (PMID: |
| | | 29721373) |
| | cMAb U36 | Sandstrom et al., Int. J. Oncol., 40(5): 1525-32 (2012) (PMID: |
| | | 22307465) |
| NKG2D | scFv | Yang et al., J. Immunother. Cancer, 7(1): 171 (2019) (PMID: 31288857) |
| | NKG2Dg scFv | Parihar et al., Cancer Immunol. Res., 7(3): 363-375 (2019) (PMID: |
| | | 30651290) |
| CD38 | Nanobody CD38 | An et al., Mol. Pharm., 15(10): 4577-4588 (2018) (PMID: 30185037) |
| | Humanized scFv | Yoshida et al., Clin. Transl. Immunology, 5(12): e116 (2016) (PMID: |
| | | 28090317) |
| GPRC5D | Humanized scFv | Smith et al., Sci. Transl. Med., 11(485)eaau7746 (2019) (PMID: |
| | | 30918115) |
| CD79b | scFv (L-H) and (H-L) | Ormhoj et al., Clin. Cancer Res., (2019) (PMID: 31439577) |
| CD103 | M290 | Zhang et al., Am. J. Transplant., 9(9): 2012-23 (2009) (PMID: |
| | | 19645708) |
| FAP | FAP5 | Wang et al., Cancer Immunol. Res., 2(2): 154-66 (2014) (PMID: |
| | | 24778279) |
| CD70 | Human CD70 | Park et al., Oral Oncol., 78: 145-150 (2018) (PMID: 29496042) |
| MUC16 | 4H11 | Koneru et al., Oncoimmunol., 4(3): e994446 (2015) (PMID: 25949921) |
| IL13Ra2 | IL13R | Kong et al., Clin. Cancer Res., 18(21): 5949-60 (2012) (PMID: |
| | | 22966020) |

Any appropriate method can be used to express an antigen receptor on a T cell having (e.g., engineered to have) a reduced expression level of a TNFR2 polypeptide. For example, a nucleic acid encoding an antigen receptor can be introduced into one or more T cells. In some cases, viral transduction can be used to introduce a nucleic acid encoding an antigen receptor into a non-dividing a cell. A nucleic acid encoding an antigen receptor can be introduced in a T cell using any appropriate method. In some cases, a nucleic acid encoding an antigen receptor can be introduced into a T cell by transduction (e.g., viral transduction using a retroviral vector such as a lentiviral vector) or transfection. In some cases, a nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced expression level of a TNFR2 polypeptide also expresses (e.g., is engineered to express) an antigen receptor, that T cell can be engineered to have a reduced expression level of that TNFR2 and engineered to express an antigen receptor using any appropriate method. In some cases, a T cell can be engineered to have a reduced expression level of a TNFR2 polypeptide first and engineered to express an antigen receptor second, or vice versa. In some cases, a T cell can be simultaneously engineered to have a reduced expression level of a TNFR2 polypeptide and to express an antigen receptor. For example, one or more nucleic acids used to reduce expression of a TNFR2 polypeptide (e.g., a lentiviral vector encoding at least one gRNA sequence specific to a nucleic acid encoding that TNFR2 polypeptide and at least one Cas9 nuclease or a nucleic acid encoding at least one oligonucleotide that is complementary to that TNFR2 polypeptide's mRNA) and one or more nucleic acids encoding an antigen receptor (e.g., a CAR) can be simultaneously introduced into one or more T cells. One or more nucleic acids used to reduce expression of a TNFR2 polypeptide and one or more nucleic acids encoding an antigen receptor can be introduced into one or more T cells on separate nucleic acid constructs or on a single nucleic acid construct. In some cases, one or more nucleic acids used to reduce expression of a TNFR2 polypeptide and one or more nucleic acids encoding an antigen receptor can be introduced into one or more T cells on a single nucleic acid construct. In some cases, one or more nucleic acids used to reduce expression of a TNFR2 polypeptide and one or more nucleic acids encoding an antigen receptor can be introduced ex vivo into one or more T cells. In cases where T cells are engineered ex vivo to have a reduced expression levels of a TNFR2 polypeptide and to express an antigen receptor, the T cells can be obtained from any appropriate source (e.g., a mammal such as the mammal to be treated or a donor mammal, or a cell line).

In some cases, a T cell having (e.g., engineered to have) a reduced expression level of a TNFR2 polypeptide can be stimulated. A T cell can be stimulated at the same time as being engineered to have a reduced level of a TNFR2 polypeptide or independently of being engineered to have a reduced level of a TNFR2 polypeptide. For example, one or more T cells having a reduced level of a TNFR2 polypeptide used in an adoptive cell therapy can be stimulated first, and can be engineered to have a reduced expression level of a TNFR2 polypeptide second, or vice versa. In some cases, one or more T cells having a reduced expression level of a TNFR2 polypeptide used in an adoptive cell therapy can be stimulated first, and can be engineered to have a reduced level of a TNFR2 polypeptide second. A T cell can be stimulated using any appropriate method. For example, a T cell can be stimulated by contacting the T cell with one or more CD polypeptides. Examples of molecules that can be used to stimulate a T cell include, without limitation, CD3, CD28, inducible T cell co-stimulator (ICOS), CD137, CD2, OX40, CD27, phorbol 12-myristate 13-acetate (PMA), and ionomycin. In some cases, a T cell can be stimulated prior to introducing components of a CRISPR/Cas system (e.g., a gRNA and/or a Cas nuclease) to the T cell to KO a nucleic acid encoding a TNFR2 polypeptide.

In some cases, the methods and materials provided herein can be used for generating natural killer (NK) cells (e.g., CAR NK cells) having a reduced expression level of a TNFR2 polypeptide. For example, a NK (e.g., a CAR NK cell) can be engineered to KO a nucleic acid encoding a TNFR2 polypeptide to reduce TNFR2 polypeptide expression in that NK cell (e.g., as compared to a comparable NK cell that is not engineered to KO a nucleic acid encoding a TNFR2 polypeptide).

This document also provides methods and materials involved in treating cancer. For example, one or more T cells having (e.g., engineered to have) a reduced expression level of a TNFR2 polypeptide (e.g., TNFR2 KO T cells) can be administered (e.g., in an adoptive cell therapy such as a CAR T cell therapy) to a mammal (e.g., a human) having cancer to treat the mammal. In some cases, methods of treating a mammal having cancer as described herein can reduce the number of cancer cells (e.g., cancer cells expressing a tumor antigen) within a mammal. In some cases, methods of treating a mammal having cancer as described herein can reduce the size of one or more tumors (e.g., tumors expressing a tumor antigen) within a mammal.

Any appropriate amount (e.g., number) of T cells having (e.g., engineered to have) a reduced expression level of a TNFR2 polypeptide (e.g., TNFR2 KO T cells) can be administered (e.g., in an adoptive cell therapy such as a CAR T cell therapy) to a mammal (e.g., a human) having cancer. In some cases, from about 0.5×10⁶ T cells per kg body weight of the mammal (T cells/kg) to about 10×10⁶ T cells/kg having a reduced expression level of a TNFR2 polypeptide can be administered to a mammal having cancer to treat the mammal. For example, a mammal having cancer can be administered a composition including from about 0.5×10⁶ T cells/kg to about 10×10⁶ T cells/kg.

In some cases, administering T cells having (e.g., engineered to have) a reduced expression level of a TNFR2 polypeptide (e.g., TNFR2 KO T cells) to a mammal results in little or no induction of a cytokine release syndrome (CRS). For example, administering T cells having a reduced level of a TNFR2 polypeptide to a mammal can result in little or no release of cytokines associated with CRS (e.g., CRS critical cytokines). Examples of cytokines associated with CRS include, without limitation, IL-6, G-CSF, IFN-g, IL-1B, IL-10, MCP-1, MIG, MIP, MIP 1b, TNF-a, IL-2, and perforin.

In some cases, administering T cells having (e.g., engineered to have) a reduced expression level of a TNFR2 polypeptide (e.g., TNFR2 KO T cells) to a mammal results in little or no neurotoxicity. For example, administering T cells having a reduced level of a TNFR2 polypeptide to a mammal can result in little or no differentiation and/or activation of white blood cells, the differentiation and/or activation of which, is associated with neurotoxicity. Examples of white blood cells, the differentiation and/or activation of which, is associated with neurotoxicity include, without limitation, monocytes, macrophages, T-cells, dendritic cells, microglia, astrocytes, and neutrophils.

Any appropriate mammal (e.g., a human) having a cancer can be treated as described herein. Examples of mammals that can be treated as described herein include, without limitation, humans, primates (such as monkeys), dogs, cats, horses, cows, pigs, sheep, mice, and rats. For example, a human having a cancer can be treated with one or more T cells having (e.g., engineered to have) a reduced expression level of a TNFR2 polypeptide in, for example, an adoptive T cell therapy such as a CAR T cell therapy using the methods and materials described herein.

When treating a mammal (e.g., a human) having a cancer as described herein, the cancer can be any appropriate cancer. In some cases, a cancer treated as described herein can include one or more solid tumors. In some cases, a cancer treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, diffuse large B cell lymphomas (DLBCL), Hodgkin's lymphomas, non-Hodgkin lymphomas, acute lymphoblastic leukemias (ALLs), chronic lymphocytic leukemias (CLLs), acute myeloid leukemias (AMLs), germ cell tumors, hepatocellular carcinomas, bowel cancers, lung cancers, breast cancers, ovarian cancers, melanomas, epithelial tumors, brain cancers, multiple myelomas, lung cancers, head and neck cancers, and sarcomas. For example, one or more T cells having (e.g., engineered to have) a reduced level of a TNFR2 polypeptide (e.g., TNFR2 KO T cells) can be used to treat a mammal having DLBCL. For example, one or more T cells having (e.g., engineered to have) a reduced level of a TNFR2 polypeptide (e.g., TNFR2 KO T cells) can be used to treat a mammal having ALL.

In some cases, the methods described herein can include identifying a mammal (e.g., a human) as having a cancer. Any appropriate method can be used to identify a mammal having cancer. For example, imaging techniques and biopsy techniques can be used to identify mammals (e.g., humans) having cancer.

A mammal (e.g., a human) having a cancer can be administered one or more T cells having (e.g., engineered to have) a reduced expression level of a TNFR2 polypeptide described herein. For example, one or more T cells having (e.g., engineered to have) a reduced expression level of a TNFR2 polypeptide (e.g., TNFR2 KO T cells) can be used in an adoptive T cell therapy (e.g., a CAR T cell therapy) to treat a mammal having a cancer. For example, one or more T cells having a reduced level of a TNFR2 polypeptide can be used in an adoptive T cell therapy (e.g., a CAR T cell therapy) targeting any appropriate antigen within a mammal (e.g., a mammal having cancer). In some cases, an antigen can be a tumor-associated antigen (e.g., an antigenic substance produced by a cancer cell). Examples of tumor-associated antigens that can be targeted by an adoptive T cell therapy provided herein include, without limitation, CD19 (associated with DLBCL, ALL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), and c-Met (associated with breast cancer). For example, one or more T cells having a reduced level of a TNFR2 polypeptide can be used in CAR T cell therapy targeting CD19 (e.g., a CART19 cell therapy) to treat cancer as described herein.

In some cases, one or more T cells having (e.g., engineered to have) a reduced expression level of a TNFR2 polypeptide (e.g., TNFR2 KO T cells) can be used in an adoptive T cell therapy (e.g., a CAR T cell therapy) to treat a mammal having a disease or disorder other than cancer. For example, one or more T cells having a reduced level of a TNFR2 polypeptide can be used in an adoptive T cell therapy (e.g., a CAR T cell therapy) targeting any appropriate disease-associated antigen (e.g., an antigenic substance produced by cell affected by a particular disease) within a mammal. Examples of disease-associated antigens that can be targeted by an adoptive T cell therapy provided herein include, without limitation epithelial antigens (e.g., colon integrins) (associated with colitis), effector T cell antigens (associated with autoimmune diseases), B cell receptor antigens (associated with antibody mediated autoimmune diseases such as lupus and pemphigus), and fungal antigens (associated with invasive fungal infections).

In some cases, one or more T cells having (e.g., engineered to have) a reduced expression level of a TNFR2 polypeptide (e.g., TNFR2 KO T cells) used in an adoptive T cell therapy (e.g., a CAR T cell therapy) can be administered to a mammal having a cancer as a combination therapy with one or more additional agents used to treat a cancer. For example, one or more T cells having a reduced level of a TNFR2 polypeptide used in an adoptive cell therapy can be administered to a mammal in combination with one or more anti-cancer treatments (e.g., surgery, radiation therapy, chemotherapy (e.g., alkylating agents such as busulfan), and/or targeted therapies (e.g., TNFR2 inhibiting agents such as monoclonal antibodies against TNFR2). In cases where one or more T cells having a reduced level of a TNFR2 polypeptide used in an adoptive cell therapy are used with additional agents treat a cancer, the one or more additional agents can be administered at the same time or independently. In some cases, one or more T cells having a reduced level of a TNFR2 polypeptide used in an adoptive cell therapy can be administered first, and the one or more additional agents administered second, or vice versa.

The invention will be further described in the following examples, which do not limit the scope of the invention described in the claims.

### EXAMPLES

### Example 1: Impact of Baseline CAR T Cell Activation on CAR T Cell Activity

It has become increasingly apparent that CAR T cells are susceptible to activation induce cell death, and that the level of CAR T cell activation is an important determinant of their fates.

This Example describes measurement of baseline levels of CAR T cell activation, determination of how CAR T cell activation impacts response in the clinic, and identifies potentially modifiable targets to favorably improve CAR T cell activation and anti-tumor activity.

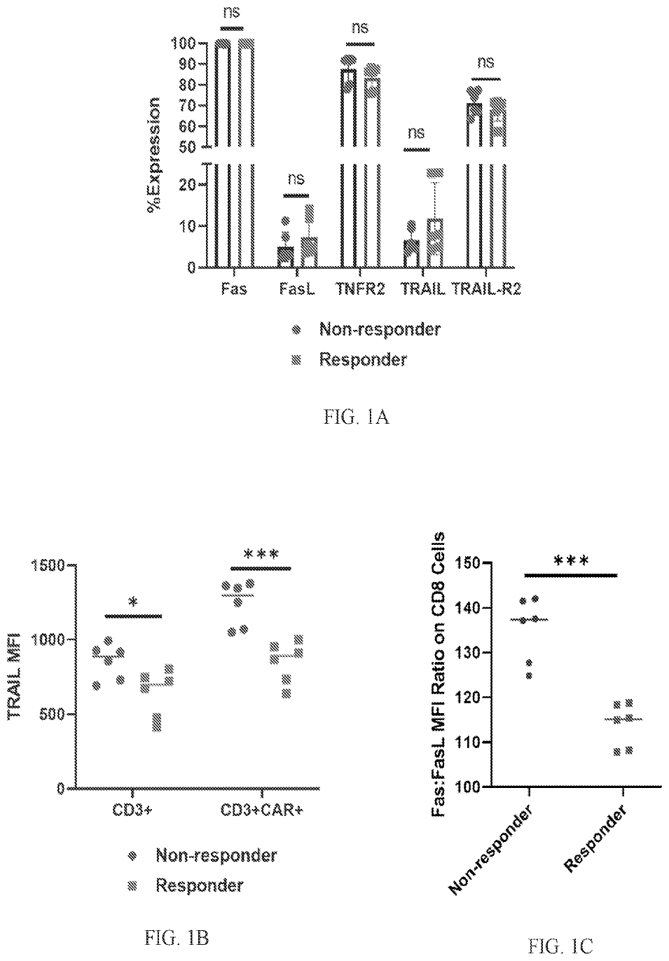

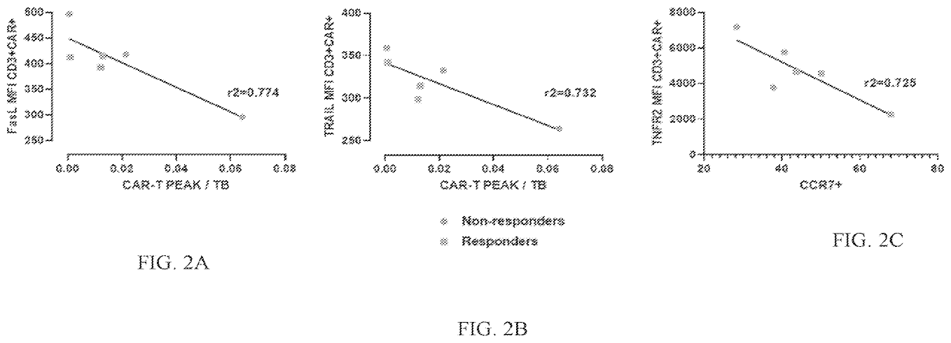

Clinically annotated CART19 Axi-Cel products for patients with large B cell lymphoma treated on the Zuma-1 clinical trial that led to the FDA approval of Axi-Cel were used (Neelapu et al., New Engl. J. Med., 377(26):2531-2544 (2017)). First, comprehensive immunophenotypic analysis was performed of death receptors in baseline Axi-Cel products (prior to infusion) comparing patients who achieved a complete response as a best response post Axi-Cel therapy (responders), to patients that achieved stable or progressive disease (non-responders). There was no difference in the expression level of TRAIL, TRAIL receptors, FAS, FasL, or TNF receptors on resting CART19 cells between responders and non-responders (FIG. 1A). However, there was a significantly higher expression of death receptors in non-responders upon their ex vivo antigen specific activation of CART19 with irradiated CD19⁺ cell lines (FIGS. 1B and 1C). This suggested that higher CAR T cell activation levels following antigen specific stimulation is associated with poor response to therapy. Furthermore, there was a significant association between an activated phenotype (higher levels of expression of TRAIL, TRAILR, Fas, FasL, and TNFR) with more terminally differentiated CAR T cells (CCR7 CAR T cells, FIGS. 2A-2C).

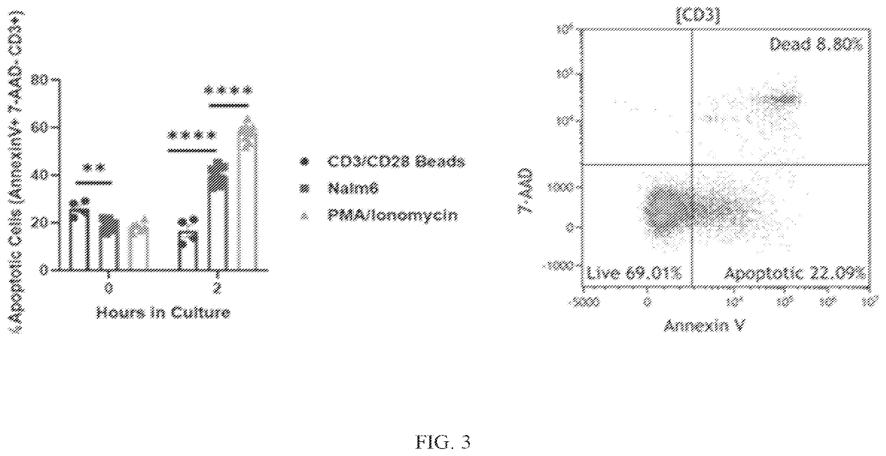

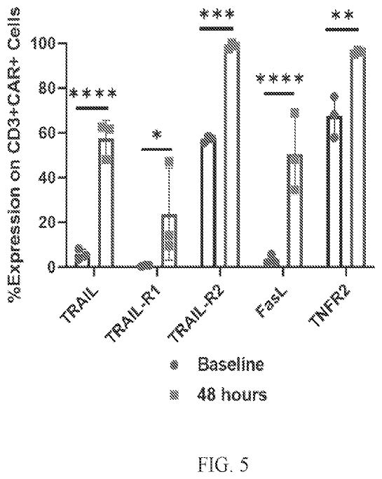

Having demonstrated an association between higher levels of activation with poor response to CAR T cell therapy, CAR T cell activation was studied in vitro and potentially modifiable targets were identified. CART19 were generated and their apoptosis was measured using an Anexin assay. While non-specific TCR activation of CART19 cells protected them from apoptosis, antigen specific activation through the CAR resulted in significant apoptosis within 2-6 hours of activation (FIG. 3). Activated CART19 upregulated Fas, FasL, TRAIL, and TRAIL R (FIG. 5).

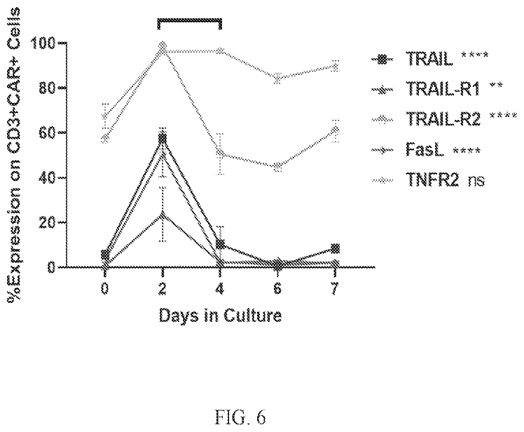

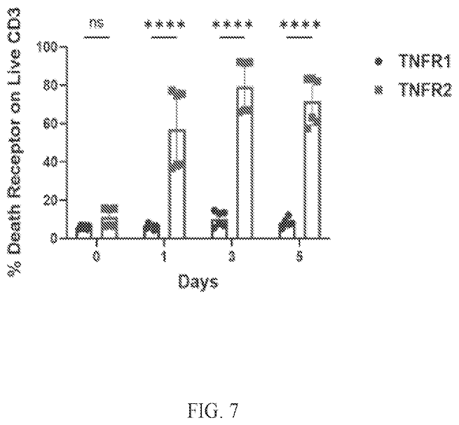

A strategy was identified to modulate CAR T cell activation to prevent their early apoptosis and enhance their anti-tumor activity. TNF receptors were therefore focused on. It was found that CART19 upregulate TNFR2, but not TNFR1 upon their antigen specific stimulation (FIG. 7). In an extended in vitro culture, where CAR T cells were repeated stimulated through the CAR to induce dysfunction, TNFR2, unlike other death receptors, was persistently elevated (FIG. 6). Collectively, these findings suggest that down regulation of TNFR2 reduces CAR T cell early activation, prevents apoptosis, and enhances their activity.

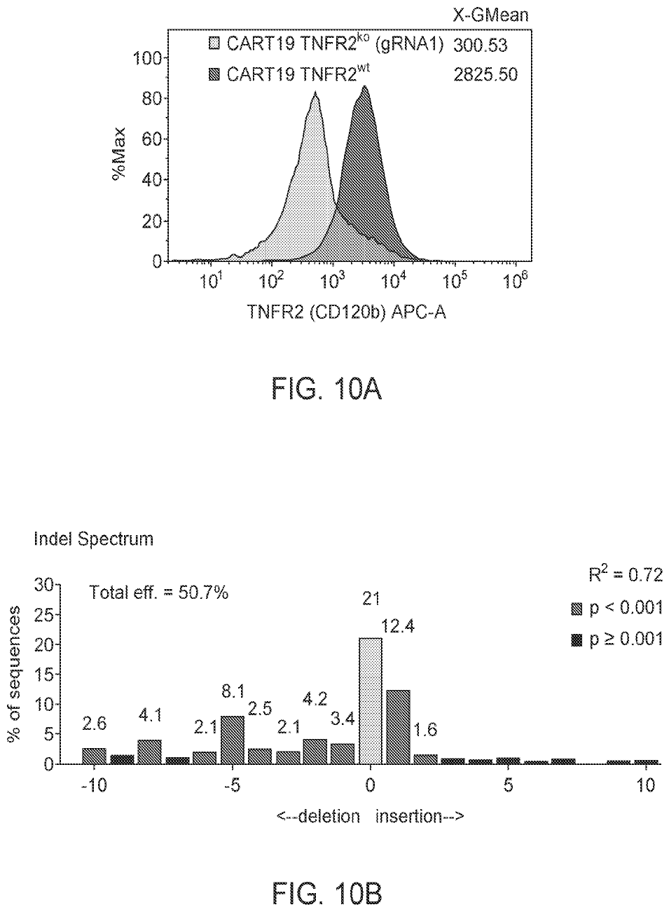

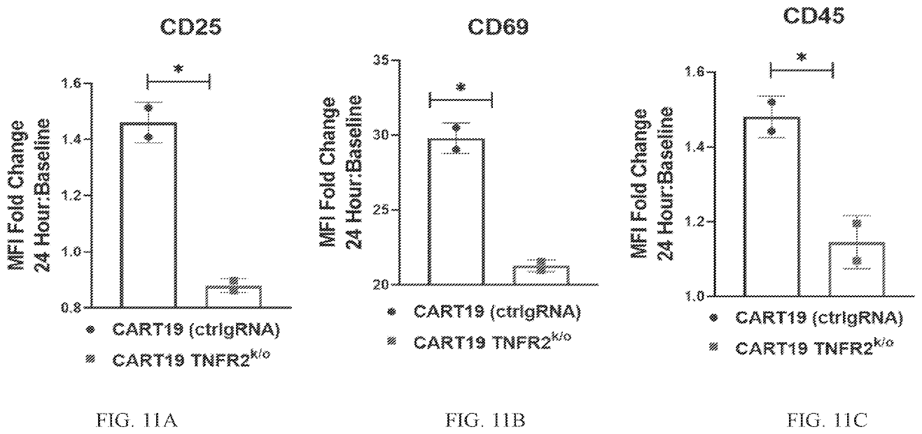

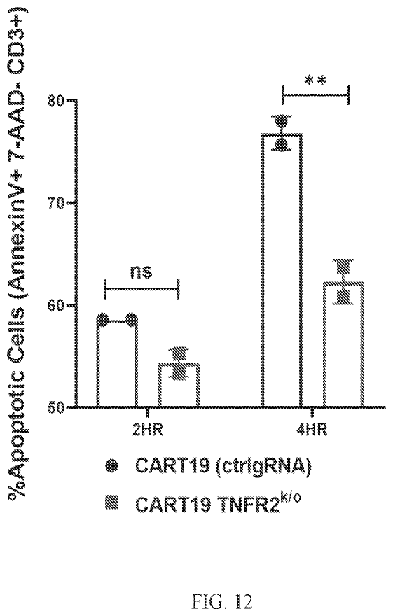

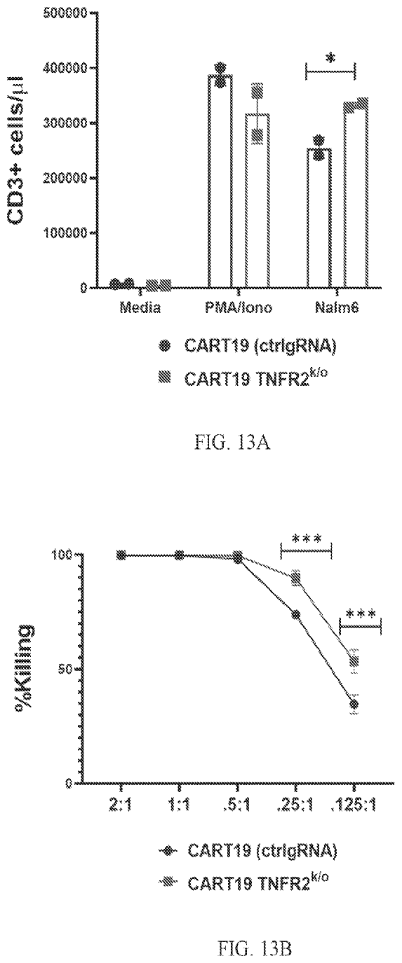

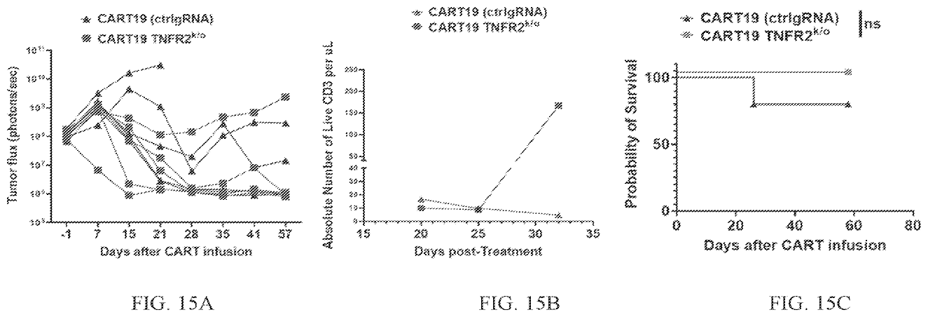

CRISPR/Cas9 was employed and a guide RNA was designed to target exon 2 of TNFR2. Using CRISPR/Cas9 during CAR T cell manufacturing, TNFR2KO CART19 cells were generated and reduced expression levels of TNFR2 were demonstrated in activated CAR T cells compared to control CART19 cells (FIG. 10A). TNFR2^{KO} CART19 cells demonstrated significantly less early activation compared to control CART19 (irrelevant gRNA CRISPR/Cas9 CART19 cells), as evident by reduction in CD25, and CD69 (FIGS. 11A and 11B), less apoptosis as measured by annexin 5 (FIG. 12), and enhanced antigen specific proliferation (FIG. 13A). Finally, in a xenograft model for relapsed CD19+ lymphoma, TNFR2^{KO} CART19 resulted in reduced initial activation, enhanced antitumor activity and survival compared to control CART19 cells (FIGS. 415A-15C). Furthermore, TNFR2^{KO} CART19 cells were more resistant to inhibition by BCL2 inhibitors compared to control CART19 cells.

In summary, these results indicate that CART19 activation level at baseline impact the response to therapy, and demonstrate that TNFR2 edited CART19 can be used to modulate CART19 activation and enhance their antitumor activity.

### Example 2: TNFR2^{KO} CART19 Development and Assessment

This Example identifies TNFR2 as a polypeptide that is involved in apoptosis of CAR T cells and in limiting CAR T cell antitumor activity.

### Results

### Levels of Activation-Induced Surface Death Receptors and Ligands on CAR T Cells

A comprehensive immunophenotypic analysis of death receptors was performed in baseline Axi-Cel products (prior to infusion) comparing patients who achieved a complete response as a best response post Axi-Cel therapy (responders), to patients that achieved stable or progressive disease (non-responders). There was no difference in the expression level of TRAIL, TRAIL receptors, FAS, FasL, or TNF receptors on resting CART19 cells between responders and non-responders. Two technical replicates and three biological replicates gated on CD3⁺ were performed. No significant differences were observed in the expression of death receptors TRAIL, FAS and TNF on resting CAR T cells between responders and non-responders from the Zuma-1 clinical trial (FIG. 1A).

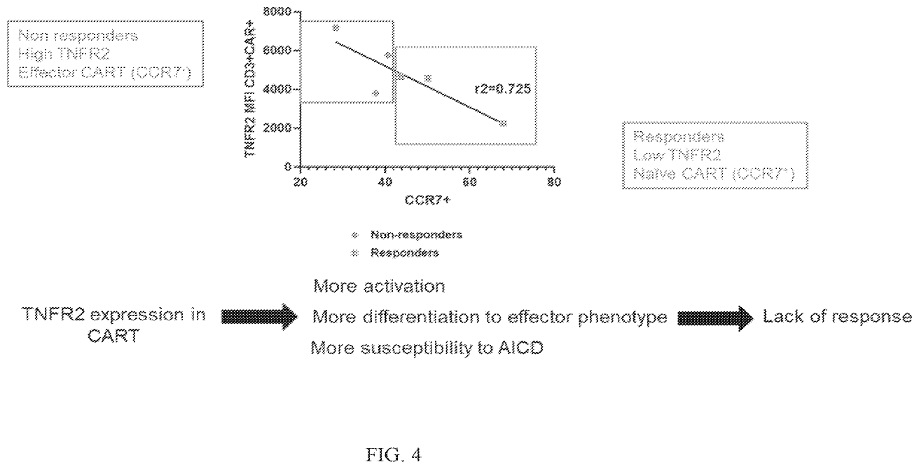

Ex vivo stimulation of Axi-Cel products (responders and non-responders) with CD19⁺ target cells (Nalm6 cell line) was performed. After 24 hours, flow cytometry was performed to assess expression of death receptors. Two technical replicates and three biological replicates were performed. Activated CAR T cells with CD19⁺ cell lines from non-responders had higher levels of expression of death receptors in comparison to non-responders from the Zuma-1 clinical trial (FIGS. 1B and 1C). There was a strong association between an activated phenotype (characterized by high levels of Fas-L, TRAIL-L and TNFR2 expression) with more terminally differentiated CAR T cells (low levels of CCR7) in non-responders from the Zuma-1 clinical trial (FIGS. 2A-2C). Together these results demonstrate a strong association between TNFR2 expression, effector CAR T phenotype, and lack of response (FIG. 4).

CART19 cells from healthy donors were co-cultured with CD19⁺ cell line Nalm6 and flow cytometry was performed at baseline and after 48 hours in order to measure the expression of death receptors and ligands (TRAIL, TNFR2 and Fas). Activated CART19 upregulated Fas, FasL, TRAIL, TRAIL-R and TNFR2 (FIG. 5).

TNFR2, but not other death receptors, was persistently elevated on CART19 cells from healthy donors using an extended in vitro culture model (FIG. 6).

TNFR1, but not TNFR2, was highly upregulated upon CART19 antigen specific stimulation (FIG. 11).

### CAR T Cell Activation Impacts Fitness and Clinical Responses

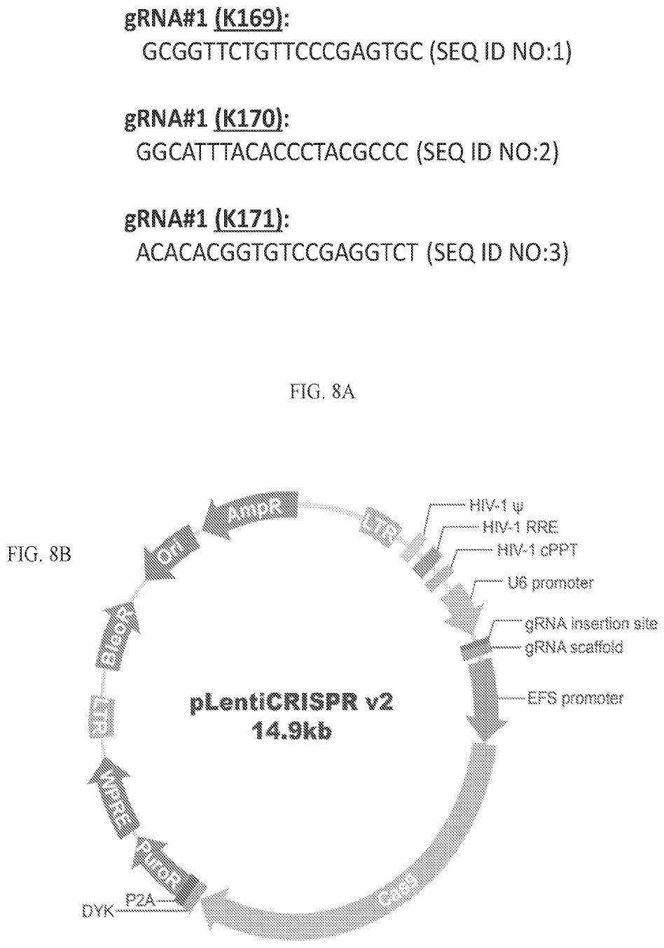

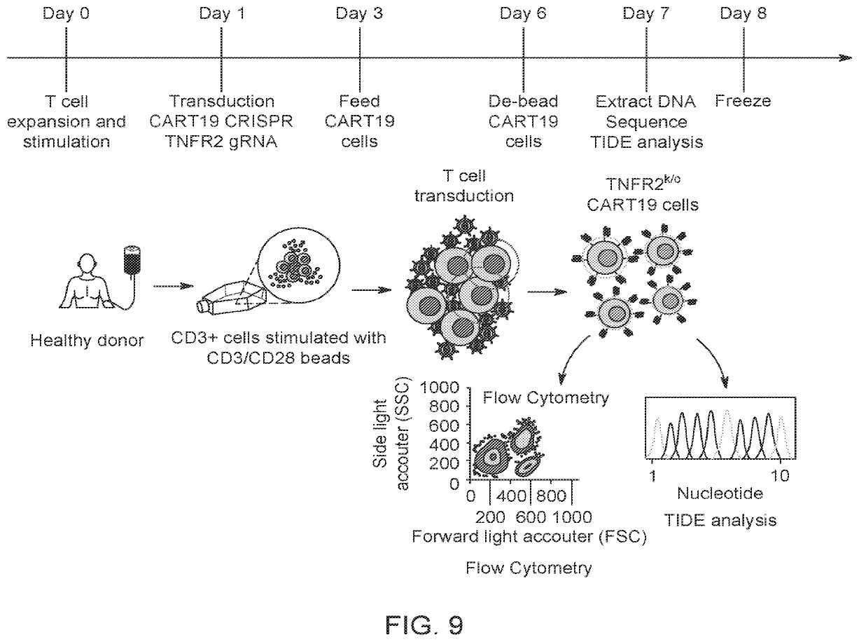

TNFR2^{k/o} CART19 cells were generated as shown in FIG. 9. TNFR2 was depleted in CART19 cells using a CRISPR/Cas9 system. Exemplary nucleic acid sequences that encode a gRNA targeting TNFR2 are shown in FIG. 8A, and an exemplary viral vector that contains a nucleic acid sequence that encodes a gRNA and a nucleic acid sequence that encodes a Cas9 nuclease is shown in FIG. 8B. Depletion of TNFR2 polypeptides in CART19 cells using CRISPR/Cas9 is demonstrated in FIGS. 10A and 10B.

Apoptosis (Annexin⁺, 7AAD″, CD3⁺) was assessed by flow cytometry analysis at different timepoints (2 hours and 4 hours). CAR T cells were cultured with irradiated CD19⁺ cells (Nalm6). TNFR2^{k/o} CART19 cells showed decreased levels of apoptosis in comparison to CART19 (ctrlgRNA) cells (FIG. 12).

Expression of activation markers (CD45, CD69 and CD25) was assessed via flow cytometry at 0 hours and 24 hours and fold change ratio was calculated. CAR T cells were cultured with CD19⁺ cells (Nalm6). TNFR2^{k/o} CART19 cells showed decreased levels of T cell activation markers in comparison to CART19 (ctrlgRNA) cells (FIGS. 11A-11C).

Antigen specific proliferation and cytotoxicity were enhanced in TNFR2^{k/o} CART19 in comparison to TNFR2^{wt}CART19 (FIGS. 13A and 13B).

### Cell-Based Targets to Modulate CAR T Cell Activation, Apoptosis, and Cytotoxicity to Improve Anti-Tumor Activity

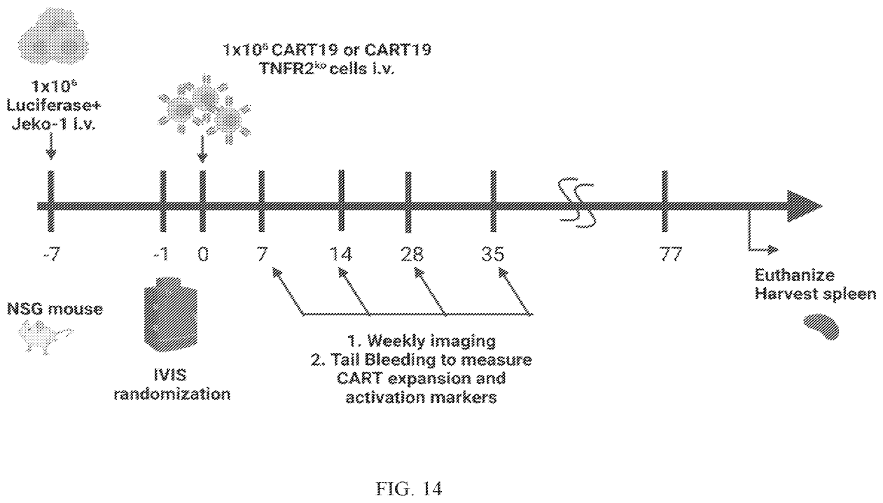

In vivo xenograft models were used to comparing CART19 and CART19 TNFR2^{ko} cells as shown in FIG. 14.

TNFR2^{k/o} CART19 cells showed improved CAR T cell expansion, enhanced anti-tumor activity and proliferation in vivo in comparison to CART19 (ctrlgRNA) cells (FIGS. 15A-15C).

Together these results demonstrate that TNFR2 KO CAR T cells are less likely to undergo apoptosis (e.g., as compared to CAR T cells that are not engineered to KO a nucleic acid encoding a TNFR2 polypeptide). These results also demonstrate that TNFR2 KO CAR T cells exhibit enhanced killing and proliferation (e.g., as compared to CAR T cells that are not engineered to KO a nucleic acid encoding a TNFR2 polypeptide).

### Materials and Methods

### Cell Lines and Clinical Samples

The following cell lines were purchased from ATCC: acute lymphoblastic leukemia cell line Nalm6 (Manassas, VA, USA) and mantle cell lymphoma cell line Jeko-1 (Manassas, VA, USA). Both cell lines were transduced with a with a firefly luciferase ZsGreen (Addgene, Cambridge, MA, USA) and then sorted to obtain >99% positive population, and they were maintained in either R10 or R20 R10 or R20 (RPMI 1640, Gibco, Gaithersburg, MD, US), 10% or 20% Fetal Bovine Serum (FBS, Millipore Sigma, Ontario, Canada), respectively, and 1% Penicillin-Streptomycin-Glutamine (Gibco, Gaithersburg, MD, US). Cell lines were kept in culture up to 20 passages, and fresh aliquots were thawed every 7-8 weeks. The use of recombinant DNA in the laboratory was approved by the Mayo Clinic Institutional Biosafety Committee (IBC). Clinically annotated CART products of patients with large B cell lymphoma from the Zuma-1 clinic trail (FDA-approved Axi-Cel) were obtained from the Kite company (Kite, a Gilead Company, Santa Monica, CA). Clinical samples were classified as patients who achieved a complete response as a best outcome (‘responders’) vs patients who achieved stable or progressive disease (‘non-responders’).

### Generation of CART19 or CART19 TNFR2^{ko} Cells

Peripheral blood mononuclear cells (PBMC) were isolated from de-identified normal donor blood apheresis cones using SepMate tubes (STEMCELL Technologies, Vancouver, Canada). T cells were separated with negative selection magnetic beads using EasySepTM Human T Cell Isolation Kit (STEMCELL Technologies, Vancouver, Canada). Primary cells were cultured in T Cell Medium made with X-Vivo 15 (Lonza, Walkersville, MD, USA) supplemented with 10% human serum albumin (Corning, NY, USA) and 1% Penicillin-Streptomycin-Glutamine (Gibco, Gaithersburg, MD, USA). CART19 cells were generated through the lentiviral transduction of normal donor T cells. Here, a second generation 4-1BB costimulated CAR construct (FMC63-41BBz) was synthesized. In addition, gRNAs targeting exon 2 of human TNFRSF1B were selected using the Broad Institute library (Sanjana et al., Nature Methods, 11(8):783-784 (2014)). The selected gRNAs were ordered in a CAS9 third generation lentivirus construct (lentiCRISPRv2), controlled under a U6 promotor (GenScript, Township, NJ, USA). Lentiviral particles were generated through the transient transfection of plasmid into 293T virus producing cells in the presence of Lipofectamine 3000 (Invitrogen, Carlsbad, CA, USA), VSV-G, and packaging plasmids (Addgene, Cambridge, MA, USA). T cells isolated from normal donors were stimulated using Cell Therapy Systems Dynabeads CD3/CD28 (Life Technologies, Oslo, Norway) at a 1:3 ratio and then transduced with lentivirus particles 24 hours after stimulation at a multiplicity of infection (MOI) of 3.0. CART cells were maintained in TCM for 5 days. At day 6, magnetic removal was performed on CART cells and cryopreserved on day 8 for future experiments. To analyze efficiency of targeting TNFR2, genomic DNA was extracted from the CART19 TNFR2^{ko} cells using PureLink Genomic DNA Mini Kit (Invitrogen, Carlsbad, CA, USA). The DNA of interest was PCR amplified using Choice Taq Blue Mastermix (Thomas Scientific, Minneapolis, MN, USA) and gel extracted using QIAquick Gel Extraction Kit (Qiagen, Germantown, MD, USA) to determine editing. PCR amplicons were sent for Eurofins sequencing (Louisville, KY, USA) and allele modification frequency was calculated using TIDE (Tracking of Indels by Decomposition) software.

### Flow Cytometric Analysis of Clinical Samples

Flow cytometric studies on ex vivo stimulated clinical samples were performed as follows. Responders (R) and Non-responders (NR) samples were stimulated with CD19+cell line Nalm6 for 24 hours. Flow cytometry was performed at baseline and 24 hours using the following antibodies: CD3 (clone SK7) APC-H7 (BD Pharmingen, San Jose, CA, USA), CD4 (clone OKT4) FITC (eBioscience, San Diego, CA, USA), CD8 (clone SK1) PerCP (BioLegend, San Diego, CA, USA), Fas L (clone NOK-1) Pe-Cy7 (BioLegend, San Diego, CA, USA), TNFR2 (clone 3G7A02) APC (BioLegend, San Diego, CA, USA), Fas (clone DX2) BV421 (BioLegend, San Diego, CA, USA), TRAIL (clone RIK-2) Pe-Cy7 (BioLegend, San Diego, CA, USA), TRAIL-R2 (clone DJR2-4) APC (BioLegend, San Diego, CA, USA) and LIVE/DEADTM Fixable Aqua Dead Cell Stain Kit (Invitrogen, Carlsbad, CA, USA). Cells were washed twice with BD stain buffer (BD Horizon, Franklin Lakes, NJ). Cytometric data were acquired using a CytoFLEX Flow Cytometer (Beckman Coulter, Chaska, MN, USA). Gating was performed using Kaluza version 2.1 (Beckman Coulter, Chaska, MN, USA).

### Apoptosis and Activation Assays

CART19 TNFR2^{wt} or CART19 TNFR2^{ko} were stimulated with PMA/ionomycin, CD19⁺ cell line Nalm6, or Cell Therapy Systems Dynabeads CD3/CD28 (Life Technologies, Oslo, Norway) at different time points (0 hours, 1 hour, 2 hours, or 4 hours). The following amounts were used for stimulation: 50 ng/ml of PMA, 1 μg/mL of ionomycin, 3:1 ratio of beads:cells, and 1:1 ratio of Nalm6:CART19. Then, cells were spun and washed with flow buffer, followed by incubation in the dark with the following reagents: CD3 (clone SK7) APC-Cy7 (560176, BioLegend, San Diego, CA, USA), Annexin V PE (BD Biosciences, San Jose, CA, USA), 7-AAD (BD Biosciences, San Jose, CA), and 1× annexin binding buffer (1:10 dilution of 10× ABB) (BD Biosciences, San Jose, CA, USA). Then, the expression of Annexin V and 7-AAD on CD3 cells was measured via flow cytometry. Similarly, for the activation assay, CART19 TNFR2^{wt} or CART19 TNFR2^{ko} were stimulated with Nalm6 CD19⁺ cell line for 24 hours. Flow cytometry was performed at baseline and 24 hours using the following antibodies: CD3 (clone OKT3) BV650 (BioLegend, San Diego, CA, USA), CD45 (clone HI30) BV421 (BioLegend, San Diego, CA, USA), CD20 (clone L27) PE (BioLegend, San Diego, CA, USA), CD25 (clone M-A251) PE-Cy7 (BD Biosciences, San Jose, CA, USA), CD69 (clone FN50) BV785 (BioLegend, San Diego, CA, USA), HLA-DR (clone L243) APC-Fire/750 (BioLegend, San Diego, CA, USA). Absolute quantification was obtained using volumetric measurement or CountBright absolute counting beads (Invitrogen, Carlsbad, CA, USA).

### T Cell Functional Assays

For proliferation assays, CART19 TNFR2^{wt} or CART19 TNFR2ko were cultured with irradiated CD19⁺ cell line Nalm6 at a 1:1 ratio or with PMA/ionomycin (Millipore Sigma, Ontario, Canada) as a positive non-specific stimulant of T cells for 5 days. Then, the cells were harvested and washed with flow buffer, following by surface staining with anti-hCD3 (eBioscience, San Diego, CA, USA) and LIVE/DEAD™ Fixable Aqua Dead Cell Stain Kit (Invitrogen, Carlsbad, CA, USA). For killing assays, the CD19⁺ NALM6 (Luciferase+) were incubated at the indicated ratios with effector T cells for 24, 48, or 72 hours as listed in the specific experiment. Killing was calculated by bioluminescence imaging on a Xenogen IVIS-200 Spectrum camera (PerkinElmer, Hopkinton, MA, USA) as a measure of residual live cells. Samples were treated with 1 μL D-luciferin (30 μg/mL) per 100 μL sample volume (Gold Biotechnology, St. Louis, MO, USA), for 10 minutes prior to imaging.

### In Vivo Studies

6-8 week old non-obese diabetic/severe combined immunodeficient mice bearing a targeted mutation in the interleukin (IL)-2 receptor gamma chain gene (NSG) female mice were purchased from Jackson Laboratories (Jackson Laboratories, Bar Harbor, ME, USA). All animal experiments were performed under an IACUC-approved protocol (A00001767). Mice were maintained in an animal barrier space that is approved by the IBC for BSL2+ level experiments (IBC #HIP00000252). Mice were intravenously injected with 1.0×10⁶ luciferase⁺ JeKo-1 cells. Fourteen days after injection, mice were imaged with a bioluminescent imager using an IVIS® Lumina S5 Imaging System (PerkinElmer, Hopkinton, MA, USA) to confirm engraftment. Imaging was performed 10 minutes after the intraperitoneal injection of 10 μL/g D-luciferin (15 mg/mL, Gold Biotechnology, St. Louis, MO, USA). Mice were then randomized based on their bioluminescence imaging to receive either untransduced T cells, CART19 TNFR2^{wt}, or CART19 TNFR2^{ko}. Serial bleeding was performed and CD3 was quantified using flow cytometry. Briefly, mouse peripheral blood was lysed using BD FACS Lyse buffer (BD Biosciences, San Jose, CA, USA) and stained with anti-human CD3 APC-Cy7 (BioLegend, San Diego, CA, USA) and anti-mouse CD45 (clone 30-F11) PE (BioLegend, San Diego, CA, USA). Absolute quantification was performed using CountBright absolute counting beads (Invitrogen, Carlsbad, CA, USA). Mice were euthanized for necropsy when moribund.

### Example 3: Treating Cancer

A human having cancer is administered CAR T cells having a reduced level of TNFR2 polypeptides (e.g., TNFR2 KO T cells).

The administered CAR T cells having a reduced level of TNFR2 polypeptides (e.g., TNFR2 KO CAR T cells) can target (e.g., target and destroy) cancer cells (e.g., cancer cells expressing a tumor antigen targeted by the CAR T cells) within a mammal.

### Example 4: Treating Cancer

T cells are obtained from a mammal having cancer and are engineered be CAR T cells having a reduced level of TNFR2 polypeptides (e.g., TNFR2 KO T cells).

The CAR T cells having a reduced level of TNFR2 polypeptides (e.g., TNFR2 KO T cells) are administered back to the human.

The administered CAR T cells having a reduced level of TNFR2 polypeptides (e.g., TNFR2 KO CAR T cells) can target (e.g., target and destroy) cancer cells (e.g., cancer cells expressing a tumor antigen targeted by the CAR T cells) within a mammal.

### Example 5: Exemplary Embodiments

Embodiment 1. A method for making a chimeric antigen receptor T cell having a reduced level of a tumor necrosis factor receptor 2 (TNFR2) polypeptide, wherein said method comprises:

(a) obtaining a T cell having endogenous alleles encoding said TNFR2 polypeptide and expressing said TNFR2 polypeptide,

(b) disrupting at least one of said endogenous alleles encoding said TNFR2 polypeptide, thereby reducing the level of expression of said TNFR2 polypeptide by said T cell, and

(c) introducing nucleic acid encoding a chimeric antigen receptor into said T cell,

wherein the resulting T cell is said chimeric antigen receptor T cell having a reduced level of TNFR2 polypeptide.

Embodiment 2. The method of embodiment 1, wherein step (b) is performed before step (c).
Embodiment 3. The method of embodiment 1, wherein step (c) is performed before step (b).
Embodiment 4. The method of any one of embodiments 1-3, wherein step (b) comprises disrupting both endogenous alleles.
Embodiment 5. The method of any one of embodiments 1-4, wherein said T cell is obtained from a human.
Embodiment 6. The method of any one of embodiments 1-5, wherein step (b) is performed ex vivo.
Embodiment 7. The method of any one of embodiments 1-5, wherein step (c) is performed ex vivo.
Embodiment 8. The method of any one of embodiments 1-5, wherein step (b) and step (c) are both performed ex vivo.
Embodiment 9. The method of any one of embodiments 1-8, wherein said chimeric antigen receptor targets a tumor-associated antigen.
Embodiment 10. The method of embodiment 9, wherein said tumor-associated antigen is CD19.
Embodiment 11. A method for making a chimeric antigen receptor T cell having a reduced level of a TNFR2 polypeptide, wherein said method comprises:

(a) obtaining a T cell (i) having endogenous alleles encoding said TNFR2 polypeptide, (ii) expressing said TNFR2 polypeptide, and (iii) expressing a chimeric antigen receptor, and

(b) disrupting at least one of said endogenous alleles encoding said TNFR2 polypeptide, thereby reducing the level of expression of said TNFR2 polypeptide by said T cell,

wherein the resulting T cell is said chimeric antigen receptor T cell having a reduced level of TNFR2 polypeptide.
Embodiment 12. The method of embodiment 11, wherein said T cell is obtained from a human.
Embodiment 13. The method of any one of embodiments 11-12, wherein step (b) comprises disrupting both endogenous alleles.
Embodiment 14. The method of any one of embodiments 11-13, wherein step (b) is performed ex vivo.
Embodiment 15. The method of any one of embodiments 11-14, wherein said chimeric antigen receptor targets a tumor-associated antigen.
Embodiment 16. The method of embodiment 15, wherein said tumor-associated antigen is CD19.
Embodiment 17. A method for making a chimeric antigen receptor T cell having a reduced level of a TNFR2 polypeptide, wherein said method comprises:

(a) obtaining a T cell (i) having a disruption in at least one endogenous allele encoding said TNFR2 polypeptide and (ii) expressing a reduced level of said TNFR2 polypeptide as compared to a comparable T cell lacking said disruption, and

(b) introducing nucleic acid encoding a chimeric antigen receptor into said T cell,

wherein the resulting T cell is said chimeric antigen receptor T cell having a reduced level of TNFR2 polypeptide.

Embodiment 18. The method of embodiment 17, wherein said T cell is obtained from a human.
Embodiment 19. The method of any one of embodiments 17-18, wherein said T cell comprises a disruption in both endogenous alleles.
Embodiment 20. The method of any one of embodiments 17-19, wherein step (b) is performed ex vivo.
Embodiment 21. The method of any one of embodiments 17-20, wherein said chimeric antigen receptor targets a tumor-associated antigen.
Embodiment 22. The method of embodiment 21, wherein said tumor-associated antigen is CD19.
Embodiment 23. A method for making a chimeric antigen receptor T cell having a reduced level of a TNFR2 polypeptide, said method comprising:

introducing a nucleic acid construct into a T cell ex vivo, wherein said nucleic acid construct comprises: a) a nucleic acid encoding a guide RNA, wherein said guide RNA is complementary to a messenger RNA encoding said TNFR2 polypeptide; b) a nucleic acid encoding a Cas nuclease, and c) a nucleic acid encoding a chimeric antigen receptor.

Embodiment 24. The method of embodiment 23, wherein said guide RNA is encoded by a nucleic acid sequence set forth in any one of SEQ ID NOs:1-6.
Embodiment 25. The method of any one of embodiments 23-24, wherein said Cas nuclease is Cas9 nuclease.
Embodiment 26. The method of any one of embodiments 23-25, wherein said nucleic acid construct is a viral vector.
Embodiment 27. The method of embodiments 26, wherein said viral vector is a lentiviral vector.
Embodiment 28. The method of any one of embodiments 23-27, wherein said chimeric antigen receptor targets a tumor-associated antigen.
Embodiment 29. The method of embodiment 28, wherein said tumor-associated antigen is CD19.
Embodiment 30. The method of any one of embodiments 23-29, wherein said introducing step comprises transduction.
Embodiment 31. A method for making a chimeric antigen receptor T cell having a reduced level of a TNFR2 polypeptide, said method comprising:

introducing a complex into a T cell ex vivo, wherein said complex comprises: a) a guide RNA, wherein said guide RNA is complementary to a messenger RNA encoding said TNFR2 polypeptide; and b) a Cas nuclease; and

introducing a nucleic acid encoding a chimeric antigen receptor into said T cell ex vivo.

Embodiment 32. The method of embodiment 31, wherein said Cas nuclease is Cas9 nuclease.
Embodiment 33. The method of any one of embodiments 31-32, wherein said complex is a ribonucleoprotein.
Embodiment 34. The method of any one of embodiments 31-33, wherein said chimeric antigen receptor targets a tumor-associated antigen.
Embodiment 35. The method of embodiment 34, wherein said tumor-associated antigen is CD19.
Embodiment 36. The method of any one of embodiments 31-35, wherein said introducing steps comprises electroporation.
Embodiment 37. A T cell comprising (a) a disruption in at least one endogenous allele encoding a TNFR2 polypeptide and (b) nucleic acid encoding a chimeric antigen receptor, wherein said T cell expresses a reduced level of said TNFR2 polypeptide as compared to a comparable T cell lacking said disruption, and wherein said T cell expresses said chimeric antigen receptor.
Embodiment 38. The T cell of embodiment 37, wherein said T cell is obtained from a human.
Embodiment 39. The T cell of any one of embodiments 37-38, wherein said T cell comprises a disruption in both endogenous alleles.
Embodiment 40. The T cell of any one of embodiments 37-39, wherein said chimeric antigen receptor targets a tumor-associated antigen.
Embodiment 41. The T cell of embodiment 40, wherein said tumor-associated antigen is CD19.
Embodiment 42. The T cell of any one of embodiments 37-41, wherein said T cell has improved antitumor activity as compared to said comparable T cell lacking said disruption.
Embodiment 43. A method for treating a mammal having cancer, wherein said method comprises administering, to said mammal, a composition comprising a T cell as set forth in any one of embodiments 37-42.
Embodiment 44. The method of embodiment 43, wherein said composition comprises from about 0.5×10⁶ to 10×10⁶ of said T cells per kg body weight of said mammal.
Embodiment 45. The method of any one of embodiments 43-44, wherein said mammal is a human.
Embodiment 46. The method of any one of embodiments 43-45, wherein said cancer is a lymphoma.
Embodiment 47. The method of embodiment 46, wherein said lymphoma is a diffuse large B cell lymphoma.
Embodiment 48. The method of any one of embodiments 43-35, wherein said cancer is a leukemia.
Embodiment 49. The method of embodiment 48, wherein said leukemia is an acute lymphoblastic leukemia.
Embodiment 50. The method of any one of embodiments 43-49, wherein said chimeric antigen receptor targets a tumor-associated antigen.
Embodiment 51. The method of embodiment 50, wherein said tumor-associated antigen is CD19.
Embodiment 52. A method for treating a mammal having cancer, wherein said method comprises administering chimeric antigen receptor T cells having a reduced level of a TNFR2 polypeptide to said mammal.
Embodiment 53. The method of embodiment 52, wherein said mammal is a human.
Embodiment 54. The method of any one of embodiments 52-53, wherein said cancer is a lymphoma.
Embodiment 55. The method of embodiment 54, wherein said lymphoma is a diffuse large B cell lymphoma.
Embodiment 56. The method of any one of embodiments 52-53, wherein said cancer is a leukemia.
Embodiment 57. The method of embodiment 56, wherein said leukemia is an acute lymphoblastic leukemia.
Embodiment 58. The method of any one of embodiments 52-57, wherein said chimeric antigen receptor targets a tumor-associated antigen.
Embodiment 59. The method of embodiment 58, wherein said tumor-associated antigen is CD19.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for making a chimeric antigen receptor T cell having a reduced level of a tumor necrosis factor receptor 2 (TNFR2) polypeptide, wherein said method comprises:
(a) obtaining a T cell having endogenous alleles encoding said TNFR2 polypeptide and expressing said TNFR2 polypeptide,
(b) disrupting at least one of said endogenous alleles encoding said TNFR2 polypeptide, thereby reducing the level of expression of said TNFR2 polypeptide by said T cell, and
(c) introducing nucleic acid encoding a chimeric antigen receptor into said T cell,
wherein the resulting T cell is said chimeric antigen receptor T cell having a reduced level of TNFR2 polypeptide.

2. (canceled)

3. (canceled)

4. The method of claim 1, wherein step (b) is performed ex vivo.

5. The method of claim 1, wherein step (c) is performed ex vivo.

6. (canceled)

7. A method for making a chimeric antigen receptor T cell having a reduced level of a TNFR2 polypeptide, wherein said method comprises:
(a) obtaining a T cell (i) having endogenous alleles encoding said TNFR2 polypeptide, (ii) expressing said TNFR2 polypeptide, and (iii) expressing a chimeric antigen receptor, and
(b) disrupting at least one of said endogenous alleles encoding said TNFR2 polypeptide, thereby reducing the level of expression of said TNFR2 polypeptide by said T cell,
wherein the resulting T cell is said chimeric antigen receptor T cell having a reduced level of TNFR2 polypeptide.

8. (canceled)

9. (canceled)

10. A method for making a chimeric antigen receptor T cell having a reduced level of a TNFR2 polypeptide, wherein said method comprises:
(a) obtaining a T cell (i) having a disruption in at least one endogenous allele encoding said TNFR2 polypeptide and (ii) expressing a reduced level of said TNFR2 polypeptide as compared to a comparable T cell lacking said disruption, and
(b) introducing nucleic acid encoding a chimeric antigen receptor into said T cell,
wherein the resulting T cell is said chimeric antigen receptor T cell having a reduced level of TNFR2 polypeptide.

11. The method of claim 1, wherein said T cell is obtained from a human.

12. (canceled)

13. (canceled)

14. A method for making a chimeric antigen receptor T cell having a reduced level of a TNFR2 polypeptide, said method comprising:
introducing a nucleic acid construct into a T cell ex vivo, wherein said nucleic acid construct comprises: a) a nucleic acid encoding a guide RNA, wherein said guide RNA is complementary to a messenger RNA encoding said TNFR2 polypeptide; b) a nucleic acid encoding a Cas nuclease, and c) a nucleic acid encoding a chimeric antigen receptor.

15. The method of claim 14, wherein said guide RNA is encoded by a nucleic acid sequence set forth in any one of SEQ ID NOs:1-6.

16. The method of claim 14, wherein said nucleic acid construct is a viral vector.

17. The method of claim 16, wherein said viral vector is a lentiviral vector.

18. (canceled)

19. A method for making a chimeric antigen receptor T cell having a reduced level of a TNFR2 polypeptide, said method comprising:
introducing a complex into a T cell ex vivo, wherein said complex comprises: a) a guide RNA, wherein said guide RNA is complementary to a messenger RNA encoding said TNFR2 polypeptide; and b) a Cas nuclease; and
introducing a nucleic acid encoding a chimeric antigen receptor into said T cell ex vivo.

20. The method of claim 14, wherein said Cas nuclease is Cas9 nuclease.

21. The method of claim 19, wherein said complex is a ribonucleoprotein.

22. The method of claim 19, wherein said introducing steps comprises electroporation.

23. A T cell comprising (a) a disruption in at least one endogenous allele encoding a TNFR2 polypeptide and (b) nucleic acid encoding a chimeric antigen receptor, wherein said T cell expresses a reduced level of said TNFR2 polypeptide as compared to a comparable T cell lacking said disruption, and wherein said T cell expresses said chimeric antigen receptor.

24. The T cell of claim 23, wherein said T cell is obtained from a human.

25. The T cell of claim 23, wherein said T cell comprises a disruption in both endogenous alleles.

26. The T cell of claim 1, wherein said chimeric antigen receptor targets a tumor-associated antigen.

27. The T cell of claim 26, wherein said tumor-associated antigen is CD19.

28. The T cell of claim 23, wherein said T cell has improved antitumor activity as compared to said comparable T cell lacking said disruption.

29. A method for treating a mammal having cancer, wherein said method comprises administering, to said mammal, a composition comprising a T cell as set forth in claim 23.

30. The method of claim 29, wherein said composition comprises from about 0.5×10⁶ to 10×10⁶ of said T cells per kg body weight of said mammal.

31. A method for treating a mammal having cancer, wherein said method comprises administering chimeric antigen receptor T cells having a reduced level of a TNFR2 polypeptide to said mammal.

32. The method of claim 29, wherein said mammal is a human.

33. The method of claim 29, wherein said cancer is a lymphoma.

34-38. (canceled)
